SEQUENCE LISTING (6 sequences)

SEQ ID NO: 1 - DNA, 40 bp, Mus musculus
  source 1..40: mol_type = genomic DNA
cagatttattactgtacattagaggtttcttgatgggttg

SEQ ID NO: 2 - DNA, 27 bp, Mus musculus
  source 1..27: mol_type = genomic DNA
aggaactccaggatggcattgggcagc

SEQ ID NO: 3 - DNA, 30 bp, Mus musculus
  source 1..30: mol_type = genomic DNA
aaacagacaaatctccagtggataaaggtt

SEQ ID NO: 4 - DNA, 32 bp, Mus musculus
  source 1..32: mol_type = genomic DNA
ttccaccaaggatacacgctaacattgatata

SEQ ID NO: 5 - DNA, 45 bp, synthetic construct
  misc_feature 1..45: note = Synthetic
  source 1..45: mol_type = other DNA
tttattactgtacattagaggtacattgatataaaatataaagtg

SEQ ID NO: 6 - DNA, 45 bp, Mus musculus
  source 1..45: mol_type = genomic DNA
tttattactgtacattagaggtttcttgatgggttgccctgaatg

(19) United States
(12) Patent Application Publication
(10) Pub. No.: US 2025/0176510 A1
(43) Pub. Date: Jun. 5, 2025

(54) BMX MOUSE MODELS FOR BECKER MUSCULAR DYSTROPHY (BMD) V

(71) Applicant: Children's National Medical Center, Washington, DC (US)

(72) Inventors: Alyson Fiorillo, Washington, DC (US); Christopher Heier, Washington, DC (US)

(21) Appl. No.: 18/843,907

(22) PCT Filed: Mar. 9, 2023
(86) PCT No.: PCT/US2023/014861

§ 371 Date: Sep. 4, 2024

Related U.S. Application Data
(60) Provisional application No. 63/318,842, filed on Mar. 11, 2022.

(51) Int. Cl. A01K 67/0276

(52) U.S. Cl. CPC A01K 67/0276; A01K 2217/075; A01K 2227/105; A01K 2267/0306

(57) ABSTRACT

The invention described herein provides a mouse model of BMD (bmx). and uses thereof. The bmx mice of the invention recapitulate several features of BMD and harbour a phenotype intermediate to healthy and Dmd null mdx52 mice. These bmx mice show deficits in muscle and cardiac function, reduced dystrophin protein in skeletal and cardiac muscle, and histopathology consistent with moderate dystrophy.

### REFERENCE TO RELATED APPLICATION

This application claims priority to and the benefit of the filing date of U.S. Provisional Patent Application No. 63/318,842, filed on Mar. 11, 2022, the entire contents of which, including any drawings and sequence listings, are incorporated herein by reference.

### BACKGROUND OF THE INVENTION

Becker muscular dystrophy (BMD) is a genetic neuromuscular disease of growing importance. It is a debilitating X-linked muscle disease caused by in-frame mutations of the dystrophin gene. These BMD-causing mutations result in production of a truncated isoform of dystrophin protein that is partially functional and expressed at reduced amounts. The reduced levels of a truncated dystrophin protein leads to progressive skeletal and cardiac muscle dysfunction.

BMD presents with reduced severity compared with Duchenne muscular dystrophy (DMD), the allelic disorder of complete dystrophin deficiency. BMD Patients have a variable presentation; some individuals show severe muscle weakness in early childhood with loss of ambulation by late teens to early 20s, whereas others remain largely asymptomatic. Ultimately, heart problems develop in most patients, with up to 50% of BMD patients eventually dying from cardiomyopathy. As a rare disease with variable pathology and no current animal model, BMD represents an understudied and underserved group with no approved therapy and very few clinical trials (two interventions in active clinical trials for BMD vs. 30 for DMD).

BMD provides hope to the Duchenne muscular dystrophy (DMD) community because it provides evidence that expressing a truncated dystrophin isoform can result in a milder disease. DMD is caused by a complete loss of functional dystrophin and is more severe with an earlier onset. A promising area of therapeutics seeks to restore expression of “Beckerlike” isoforms of dystrophin through mRNA-splice modulating antisense oligos (AOs). Preclinical dystrophinopathy research has focused primarily on DMD because there are several available mouse models that recreate dystrophin-null mutations. These include mdx23, mdx52 and several CRISPR mouse and rat models.

Importantly, studies in mdx mice have led to development and accelerated approval of four exon-skipping AO drugs for DMD (eteplirsen, golodirsen, viltolarsen and casimersen). Although these dystrophin restoration therapies are promising, they are not curative. In the best-case scenario, these therapeutics would convert a DMD genotype into a milder BMD phenotype. BMD therefore serves as a model for efficient dystrophin restoration therapies, and as these therapies enter widespread clinical use, there will be a further need for development of BMD therapeutics and disease models.

BMD, however, is understudied and underserved—there are no pharmacological treatments and have been few clinical trials. The discordance between therapeutic efforts is likely due, in part, to the absence of a mouse model of BMD.

### SUMMARY OF THE INVENTION

The invention described herein provides a mouse model of BMD, which 1) enables a greater understanding of disease pathophysiology, and 2) de-risks potential therapeutics pathways well before first-in-human trials. Importantly, the subject BMD mouse model is beneficial for numerous dystrophin restoration therapies in research and development and clinical trials, which convert a DMD genotype to a BMD phenotype.

In one aspect, the invention provides a mouse comprising an artificially (e.g., in-frame) deleted (e.g., not naturally existing) dystrophin/Dmd gene, such as completely or partially lacking exons 45-47 of a wild-type Dmd gene, wherein said artificially deleted dystrophin/Dmd gene leads to a BMD phenotype.

In certain embodiments, the artificially deleted Dmd gene corresponds to the most common BMD mutation in human.

In certain embodiments, the artificially deleted Dmd gene comprises a deletion of about 40,000 bp of genomic DNA sequence.

In certain embodiments, the artificially deleted Dmd gene comprises a deletions starting at exon 45, such as: 1) an exon 45-48 deletion; 2) an exon 45-49 deletion; 3) an exon 45-51 deletion; 4) an exon 45-53 deletion; or, 5) an exon 45-55 deletion.

In certain embodiments, the artificially deleted Dmd gene comprises a deletion of a single exon, such as: 1) an exon 48 deletion, or, 2) any single exon deletion between exon 23 and exon 42.

In certain embodiments, the artificially deleted Dmd gene comprises an in-frame deletion a hot-spot region exon selected from: exon 43-44, exon 48-51, exon 48-53, exon 49-51, exon 49-53, exon 50-51, exon 51-52, exon 52-53, and exon 52-55.

In certain embodiments, the artificially deleted Dmd gene is created via a DNase (e.g., a DNase capable of or designed for deleting said exons 45-47).

In certain embodiments, the DNase is a CRISPR/Cas effector enzyme (e.g., a Class 2, Type II enzyme such as Cas9, or a Class 2, Type V enzyme such as Cas12a/Cpf1, Cas12b, Cas12c, Cas12d, cas12e, Cas12f, Cas12g, Cas12h, Cas12i or Cas12k); a meganuclease, a ZFN (zinc finger nuclease), a TALEN (Transcription Activator-Like Effector Nuclease), an ARCUT (Artificial Restriction DNA Cutteror), or a Fok-dCas nuclease.

In certain embodiments, the mouse exhibits a phenotype intermediate to WT and mdx mice at the functional, molecular, and histological level.

In certain embodiments, the mouse shows impaired motor function, such as reduced forelimb and hindlimb grip strength (e.g., −15% or −39%), wire hang time, and/or in vivo isometric torque (e.g., −10%).

In certain embodiments, muscles from the mouse have increased myofiber size variability (minimal Feret's diameter) and centrally located nuclei that indicate of degeneration/regeneration.

In certain embodiments, muscles from the mouse have moderately increased levels of inflammatory/necrotic foci, collagen deposition (e.g., +1.4-fold or more) and/or trends of sarcolemmal damage as measured by the intracellular presence of IgM.

In certain embodiments, muscles from the mouse have reduced dystrophin protein levels (e.g., ˜20-50% of WT levels) in skeletal and cardiac muscles, and/or increased expression of miRNAs that target the 3′ UTR of Dmd transcripts; optionally, Dmd transcript levels are unchanged.

In certain embodiments, muscles from the mouse show increased expression of NF-kB-driven inflammatory genes and miRNAs, and genes indicative of active fibrosis.

In certain embodiments, serum from the mouse shows increased levels of creatine kinase (CK) compared to wt mouse, indicative of muscle damage.

In certain embodiments, the mouse shows signs of cardiomyopathy or decreased heart function compared to wt mouse, indicative of heart damage.

In certain embodiments, the mouse is homozygous/hemizygous.

In certain embodiments, the mouse is a male.

In certain embodiments, the mouse is a female.

In certain embodiments, the mouse is aged/aging (e.g., at least 3 months, 6 months, 9 months, 12 months, 15 months, 18 months, 21 months, 24 months, 27 months, or 30 months).

Another aspect of the invention provides a muscle tissue of any one of the mouse of the invention.

In certain embodiments, the muscle tissue is a skeletal muscle tissue.

In certain embodiments, the muscle tissue is a cardiac muscle tissue.

In certain embodiments, the muscle tissue is a smooth muscle tissue.

Another aspect of the invention provides a muscle cell of/from any one of the mouse of the invention.

In certain embodiments, the muscle cell is a primary cell.

In certain embodiments, the muscle cell is a progeny of a cultured cell.

Another aspect of the invention provides a method of characterizing BMD disease progression, the method comprising recording, analyzing, and/or characterizing at least one phenotype related to muscular dystrophy in the mouse of the invention, the muscle tissue of the invention, and/or the muscle cell of the invention.

In certain embodiments, the method further comprises applying a candidate therapy to the mouse, the muscle tissue, and/or the muscle cell.

In certain embodiments, the candidate therapy is a small molecule compound, a biologic macromolecule (e.g., antisense polynucleotide), or a viral vector (e.g., a recombinant AAV expressing a gene effective to treat muscular dystrophy such as DMD and/or BMD).

It should be noted that any embodiment of the invention as described herein, including those described only in the examples or claims, can be combined with one or more additional embodiment(s) of the invention unless such combination is improper of expressly disclaimed.

### BRIEF DESCRIPTION OF THE DRAWINGS

FIGS. 1A-1D show generation of the bmx mouse model of BMD by deletion of dystrophin exons 45-47. (FIG. 1A) Schematic of the dystrophin gene showing CRISPR/Cas9-targeted deletion of exons 45-47. (FIG. 1B) DNA sequencing showing genomic deletion of dystrophin exons 45-47. (FIG. 1C) Protein structure of dystrophin A45-47 shows disruption of the nNOS binding domain and a disruption of the rod domain that results in an out-of-phase spectrin-type repeat (STR) pattern. (FIG. 1D) mRNA levels of dystrophin exons 2-3 and exons 76-77 are unchanged in Becker muscular dystrophy (bmx) mice, whereas dystrophin exons 45-46 are deleted in quadriceps muscle. NT=amino-terminus, H=Hinge, R=STR, CR=cysteine-rich, CT=carboxy-terminus. ANOVA; n=7-8; ***P<0.001, ****P<0.0001.

FIGS. 2A-2G show that the bmx mouse has reduced motor function, muscle force and heart function. (FIG. 2A) Grip strength of bmx mice was reduced in both the forelimb (P=0.0465) and hindlimb (P=0.0002) grip strength tests. n=8. (FIG. 2B) Suspension time of bmx mice was reduced in the wire hang test (P=0.0087) and in the box hang test (P=0.0489). n=8. (FIG. 2C) In vivo maximum isometric torque and torque-frequency curve for anterior crural muscles of WT, bmx, and mdx52 mice. Maximum isometric torque was reduced in bmx mice (P=0.0110). n=6-7. (FIG. 2D) Left; ex vivo eccentric contraction-induced lengthening force drop in EDL, bmx shows increased injury (force drop) versus WT. (P=0.0249); right; force drop, expressed as a percent of initial eccentric contraction force, is shown across 10 eccentric contractions. (FIG. 2E) Echocardiography of aged (18-month-old) bmx mice shows a deficit in heart function through a decrease in fractional shortening (P=0.0036) and ejection fraction (P=0.0131). n=5-7. (FIG. 2F) Representative M-mode images of the parasternal short axis are provided. (FIG. 2G) Serum CKM levels in aged (1-year-old) mice assayed via ELISA. n=6-7. ANOVA, *P≤0.05, **P≤0.01, ***P≤0.001, ****P≤0.0001.

FIGS. 3A-3E show that the bmx mice have increased muscle mass. Body and tissue weights of age-matched WT, bmx and mdx52 mice were assayed at 5 months of age. (FIG. 3A) bmx mice show moderate increases in body mass at 5 months (P=0.0633; n=8). (FIGS. 3B & 3C) The weight of the tibialis anterior (TA; P=0.0096) and (FIG. 3C) quadriceps (P=0.0456; n=12) is significantly increased in bmx mice. (FIG. 3D) Heart weight in bmx mice is similar to that of WT mice (P=0.4288; n=8). (FIG. 3E) Spleen weight was elevated in bmx mice but was not significant (P=0.1048; n=6-8). ANOVA, *P≤0.05, **P≤0.01, ****P≤0.0001.

FIGS. 4A-4F shows that muscles from bmx mice show centrally localized nuclei and increased variation in fibre size. (FIG. 4A) Laminin immunofluorescence of WT, bmx and mdx52 gastrocnemius muscle. DAPI was used as counterstain to visualize myonuclei. Bar=100 μM. (FIG. 4B) Histogram of minimal Feret's diameter and the variance coefficient (VC) of minimal Feret's diameter. Bmx myofibres have increased variation of minimal Feret's diameter (P=0.0017; n=4). (FIG. 4C) Histogram of myofibre cross sectional area (CSA) and VC of myofibre CSA. bmx myofibres have increased variation of CSA (P=0.0182; n=4). (FIG. 4D) The percentage of centrally nucleated myofibres was increased in bmx mice (P<0.0001; n=4). (FIG. 4E) % of BrdU+ fibres in the tibialis anterior (P=0.0058; n=6). (FIG. 4F) BrdU immunofluorescence, Bar=50 μM. ANOVA, *P≤0.05, **P≤0.01, ***P≤0.001.

FIGS. 5A-5B show that Dystrophin protein levels, but not RNA, are reduced in bmx mice. (FIG. 5A) qRT-PCR showing levels of Dmd mRNA as measured by a probe specific to the exon 76-77 junction in the diaphragm, quadriceps, tibialis anterior, gastrocnemius, triceps and heart of WT, bmx and mdx52 mice. n=7-8. (FIG. 5B) Dystrophin (red) and laminin (green) immunofluorescence staining shows reduction of dystrophin in skeletal and cardiac muscles in bmx mice. DAPI was used as a counterstain to visualize myonuclei. Bar=50 μM. ANOVA, *P≤0.05, ****P 0.0001.s

FIGS. 6A-6D show quantification of dystrophin protein in bmx mice using capillary Western assay and localization of dystrophin-associated proteins. (FIG. 6A) Dystrophin protein levels were determined by capillary electrophoresis immunoassay. Depicted is a virtual Wes blot. (FIG. 6B) Wes quantification in the diaphragm (P<0.0001), triceps (P<0.0001) and heart (P<0.0001) of bmx mice. ANOVA, n=7-8. **P≤0.01, ****P≤0.0001. (FIG. 6C) nNOS immuno-fluorescence (red) was performed in the gastrocnemius muscles and shows an absence of staining for both bmx and mdx. (FIG. 6D) Immunofluorescence for the dystrophin-associated protein α-sarcoglycan (green) was performed in gastrocnemius muscles of WT, bmx and mdx52 muscles. Results show reduced staining and reduced colocalization in bmx and mdx52 at the sarcolemma. Laminin (red) was used to visualize all muscle fibres and DAPI was used as a counterstain to visualize myonuclei. Bar=50 μM.

FIGS. 7A-7F show that muscle inflammation is present in bmx mice. (FIG. 7A) Heat map of inflammatory gene expression depicting fold change in bmx over WT in the diaphragm (Dia), quadriceps (Quad), gastrocnemius (Gas), triceps (Tri) and tibialis anterior (TA). (FIG. 7B) Ccl2 and Il1b are elevated in bmx gastrocnemius muscles n=8. (FIG. 7C) Heat map of dystrophin-targeting miRNA and inflammatory miRNA expression depicting fold change in bmx over WT. (FIG. 7D) Graphs show elevated levels of dystrophin-targeting miRNAs miR-146a and miR-31 in bmx gastrocnemius muscles n=8. (FIG. 7E) Graphs show elevated levels of chronic inflammatory miRNAs miR-142-3p and miR-142-5p in bmx gastrocnemius muscles. (FIG. 7F) Haematoxylin and eosin-stained gastrocnemius of WT, bmx and mdx52 mice. Left: representative images, right: quantification of dystrophic foci in muscles showing bmx mice have an increase in inflammation and necrosis n=5. ANOVA. #P<0.10; *P<0.05; **P≤0.01, ****P≤0.0001.

FIGS. 8A-8E show markers of fibrosis and muscle damage in bmx mice. (FIG. 8A) Heat map showing relative levels of fibrosis-associated genes in all bmx skeletal muscle analysed (vs. WT). (FIG. 8B) qRT-PCR of gastrocnemius muscle from WT, bmx and mdx52 muscles showing elevated Col1a1 (P=0.0288), Col3a1 (P=0.0452) and Tnc (P=0.0273) n=8. (FIG. 8C) Trichrome staining of quadriceps muscle from WT, bmx and mdx52 mice. Bmx mice show a 41.7% increase in fibrotic staining area (P=0.0217), ANOVA. (FIG. 8D) left: Col1a1 immunofluorescence was performed in the tibialis anterior (TA) muscles and shows thickening around myofibres in bmx and mdx52 mice; right: qPCR of Col1a1 in TA muscles showed elevated expression in bmx (P=0.0085) n=8. (FIG. 8E) WT, bmx and mdx52 quadriceps were immunostained with an antibody against IgM to assess muscle damage. The bmx gastrocnemius muscles showed a 309.2% increase in IgM-positive myofibers (P=0.0878; n=3-4). ANOVA, *P≤0.05, **P≤0.01.

FIG. 9 shows that TA, heart and diaphragm of the bmx mice lacked exons 45-47, using a probe for exons Dmd 45-46 to compare expression of Dmd between WT and bmx mice. Also see FIG. 1D. Left bars: WT. Middle Bars: bmx. Right bars: mdx. ****P≤0.0001.

FIG. 10A shows modest reductions in maximal values for bmx versus WT (48.74 vs. 53.63 mN*m/kg, P=0.1235) and were significantly elevated versus mdx52 (P=0.0295), based on in vivo specific isometric torque measurements for anterior crural muscles.

FIG. 10B shows that the ex vivo EDL contraction-induced injury protocol resulted in a 42.49% drop in isometric force in bmx EDL (P=0.0307) vs. 33% isometric force drop in WT and a 38% drop in mdx EDL (FIG. 10B).

FIG. 11 shows that bmx mice has increased muscle mass in gastrocnemius (9.135% increase, P=0.0143), and triceps (17.95% increase, P=0.0583) at 5 months of age.

FIGS. 12A-12C show that bmx skeletal and heart muscles had, on average, ˜50% less dystrophin than WT (quadriceps, P=0.0064; TA, P=0.0016; gastroc, P=0.0010), based on Capillary Western immunoassays (Wes).

FIGS. 13A-13B show that a ˜2.5-fold increase in Dp71 (the shorter, C-terminal, ubiquitously expressed dystrophin isoform) expression was observed in mdx52 diaphragms (P=0.0445), while bmx mice showed slightly elevated levels of Dp71 (˜2-fold) without reaching statistical significance (P=0.126).

### DETAILED DESCRIPTION OF THE INVENTION

The invention described herein provides clinically pertinent BMD mouse models that facilitates an improved understanding of disease pathology and functionality of dystrophin isoform domains, while also providing a preclinical platform for development of the first BMD therapeutics.

More specifically, the invention described herein provides a mouse comprising an artificially (e.g., in-frame) deleted (e.g., not naturally existing) dystrophin/Dmd gene, such as completely or partially lacking exons 45-47 of a wild-type Dmd gene, wherein said artificially deleted dystrophin/Dmd gene leads to a BMD phenotype.

In certain embodiments, the artificially deleted Dmd gene is an in-frame deletion, in that the resulting DNA sequence comprises coding sequences for wt polypeptide sequences encoded by exon sequences upstream and downstream of the in-frame deletion. In certain embodiments, the resulting DNA sequence encodes a mutant mature mRNA after splicing, wherein the mutant mature mRNA encodes wt Dmd protein residues not encoded by the exons in the in-frame deletion.

In certain embodiments, the artificially deleted dystrophin/Dmd gene completely lacks exons 45-47 of the wt Dmd gene of the mouse. For example, in one embodiment, the deleted Dmd gene may lack all nucleotides of Exons 45-47, including all introns between Exon 45 and Exon 46 and between Exon 46 and Exon 47. In another embodiment, the deleted Dmd gene may lack all nucleotides of Exons 45-47, but leaves behind at least some intron sequences of introns between Exon 45 and Exon 46 and between Exon 46 and Exon 47.

In certain embodiments, each of Exons 45-47 are at least partially deleted to render them non-functional. For example, if there is any remaining exon sequences from Exons 45-47, the remaining sequences either cannot be spliced into a mature mRNA, or if spliced into the mature mRNA, fail to translate in-frame, it translated at all.

In certain embodiments, the artificially deleted Dmd gene comprises a deletions starting at exon 45, such as: 1) an exon 45-48 deletion; 2) an exon 45-49 deletion; 3) an exon 45-51 deletion; 4) an exon 45-53 deletion; or, 5) an exon 45-55 deletion.

In certain related embodiments, the artificially deleted Dmd gene comprises a deletion of a single exon, such as: 1) an exon 45, 46, 47, or 48 deletion, or, 2) any single exon deletion between exon 23 and exon 42.

In certain embodiments, the artificially deleted Dmd gene comprises an in-frame deletion a hot-spot region exon selected from: exon 43-44, exon 48-51, exon 48-53, exon 49-51, exon 49-53, exon 50-51, exon 51-52, exon 52-53, and exon 52-55.

In certain embodiments, the artificially deleted Dmd gene comprises nucleotide sequences not present in wt Dmd gene. For example, due to the specific method used to generate the deletion, DNA double stranded breaks (DSBs) may be generated initially, which are then repaired by host mouse cells to rejoin the DSBs to create a junction sequence with extra nucleotides not present in wt Dmd gene. In other embodiments, the repair of the DSBs does not introduce nucleotides not present in wt Dmd gene.

In certain embodiments, the artificially deleted Dmd gene further deletes nucleotide sequences in the intron immediately upstream of Exon 45 and/or the intron immediately downstream of Exon 47.

In certain embodiments, the artificially deleted (e.g., not naturally existing) dystrophin/Dmd gene produces a mutant mRNA transcript transcribed in vivo in substantially the same amount/level (e.g., at least about 70%, 80%, 90%, 100%, 110%, or about 120%) compared to a wt Dmd mRNA, e.g., in diaphragm, quad, TA, gastroc, triceps, and/or heart. In certain embodiments, the artificially deleted (e.g., not naturally existing) dystrophin/Dmd gene produces a mutant mRNA transcript transcribed in vivo in substantially higher amount in TA (e.g., 25-30% higher) and gastrocnemius (e.g., 50-60% higher) muscles.

In certain embodiments, mutant mRNA transcript is translated to a mutant Dmd protein

In certain embodiments, the mutant mRNA transcript does not have substantial 3′ destabilization.

In certain embodiments, the artificially deleted (e.g., not naturally existing) dystrophin/Dmd gene produces a mature mRNA that lacks functional coding sequence for a polypeptide sequence encoded by wt Exons 45-47. In certain embodiments, the mature mRNA is translated without a functional polypeptide region encoded by wt Exons 45-47. In certain embodiments, the mature mRNA is translated to produce a mutant dystrophin protein defective in nNOS binding, such as substantially lacking (e.g., less than 50%, 40%, 30%, 20%, 10%, 5%, 3%, 2%, 1% or less) nNOS binding compared to wt dystrophin. In certain embodiments, the mature mRNA is translated to produce a mutant dystrophin protein that lacks spectrin repeat R17, R18, or both (though spectrin repeats R16 and/or R19 may be substantially preserved).

In certain embodiments, the subject bmx mouse has substantially complete loss of nNOS localization at sarcolemma.

In certain embodiments, the subject bmx mouse has reduced amounts and/or reduced colocalization at myofibre membranes by α-sarcoglycan compared to wt mouse.

In some embodiments, Dystrophin protein levels are reduced in the subject bmx mice, while expression of miRNAs that target the 3′ UTR are increased.

In certain embodiments, muscles from the mouse have reduced dystrophin protein levels (e.g., ˜20-50% of WT levels) in skeletal and cardiac muscles, and/or increased expression of miRNAs that target the 3′ UTR of Dmd transcripts; optionally, Dmd transcript levels are unchanged.

In certain embodiments, Dystrophin protein levels are reduced by about 40-50% in skeletal and/or heart muscles of the subject bmx mouse compared to wt mouse.

In certain embodiments, Dp71 fragment level is significantly higher in diaphragm compared to wt mouse.

BMD patients show variability in dystrophin protein levels that partially correlate with disease severity, though even “mild” patients exhibit muscle pathology. Inter-patient and intra-patient variability is observed with respect to dystrophin protein amount. Specifically, variable and reduced dystrophin protein levels are observed in muscle biopsies from different patients, from different muscles and even within different regions of a single muscle biopsy. Elevated serum CK levels are present at birth, and up to 95% of patients can be detected by screening for serum CK levels. Muscle biopsies show increases in fibre size variability, centralized nuclei, fibre degeneration and fibre branching. Muscle atrophy and pseudohypertrophy are also seen, with fibrosis or fatty replacement of muscle.

In certain embodiments, the artificially deleted Dmd gene corresponds to the most common BMD mutation in human.

In one embodiment, the BMD mice of the invention comprises a large (e.g., about 40 kb) deletion mutation, which can be made by gene editing tools such as CRISPR technology, to remove exons 45-47 of the endogenous murine dystrophin (Dmd) gene, recreating the most common BMD-causing deletion in humans.

In certain embodiments, the artificially deleted Dmd gene is created via a DNase (e.g., a DNase capable of or designed for deleting said exons 45-47).

In certain embodiments, the DNase is a CRISPR/Cas effector enzyme (e.g., a Class 2, Type II enzyme such as Cas9, or a Class 2, Type V enzyme such as Cas12a/Cpf1, Cas12b, Cas12c, Cas12d, cas12e, Cas12f, Cas12g, Cas12h, Cas12i or Cas12k); a meganuclease, a ZFN (zinc finger nuclease), a TALEN (Transcription Activator-Like Effector Nuclease), an ARCUT (Artificial Restriction DNA Cutteror), or a Fok-dCas nuclease.

In some embodiments, the subject bmx mouse model (for Becker muscular dystrophy, X-linked) has molecular, histopathological and functional deficits consistent with BMD patients while displaying phenotypes that are intermediate to control wild-type (WT) and DMD model (mdx, such as mdx52) mice.

In some embodiments, the subject bmx mice show strength deficits, such as through motor function phenotyping and/or physiological isometric torque assays. In certain embodiments, strength deficits are assessed at 10 weeks (e.g., assessing forelimb/hindlimb grip strength), 14 weeks (e.g., assessing two-limb wire hang), and/or 15 weeks (e.g., assessing four-limb grid hang).

In certain embodiments, the subject bmx mouse has reduced grip strength for forelimb and/or hindlimb compared to wt mouse. In certain embodiments, the subject bmx mouse has reduced suspension times in wire hang and/or box hang compared to wt mouse. In certain embodiments, the subject bmx mouse has significantly reduced maximum isometric torque in the tibialis anterior (TA) compared to wt mouse (such as based on in vivo isometric torque assay).

In certain embodiments, the subject bmx mouse has significant increase in maximal in vivo specific isometric torque compared to a Dmd null mouse (such as mdx52). In certain embodiments, the subject bmx mouse has modest (e.g., about 10%) reduction in maximal in vivo specific isometric torque compared to wt mouse.

In certain embodiments, the subject bmx mouse has intermediate level of contractile performance and/or resistance to eccentric injury between wt and Dmd null mouse, for EDL muscle.

In certain embodiments, the subject bmx mouse has significant decline in a heart function measure, such as fractional shortening and ejection fraction, compared to wt mouse.

In certain embodiments, the subject bmx mouse has >10-fold, 20-fold, 30-fold, 40-fold, 50-fold, 60-fold or more serum creatine kinase muscle-type (CKM) level increase compared to wt mouse at about 18-mon old.

In certain embodiments, the subject bmx mouse has significantly lower (e.g., at least 10%, 20%, 30% lower) serum creatine kinase muscle-type (CKM) level compared to Dmd null mouse at about 18-mon old.

In certain embodiments, the subject bmx mouse has at least 5-10% increase in body and muscle mass at 5 months compared to wt mouse. In certain embodiments, the increased muscle mass is present in substantially all skeletal muscles, including TA, quadriceps, gastrocnemius, and triceps. In certain embodiments, the increased muscle mass is not present in heart mass.

In certain embodiments, the subject bmx mouse has at least 10-15% increase in spleen mass, and/or increased circulating inflammation, at 5 months compared to wt mouse.

In some embodiments, muscle sections of the subject bmx mice show increases in fiber size variability, centrally nucleated fibers, inflammation, degenerative damage, and collagen deposition.

In certain embodiments, muscles from the mouse have increased myofiber size variability (minimal Feret's diameter) and centrally located nuclei that indicate of degeneration/regeneration.

In some embodiments, muscle sections of the subject bmx mouse has increased (15-20%) number of both smaller and larger myofibres compared to wt mouse, based on, e.g., histograms plotting minimal Feret's diameter, CSA measurement, and/or variance coefficients (VCs).

In some embodiments, the subject bmx mouse has significantly increased (e.g., 5-10 times more) centrally nucleated fibres compared to wt mouse.

In certain embodiments, the subject bmx mouse has increased regeneration and myofibre hypertrophy compared to WT mouse.

In certain embodiments, the subject bmx mouse has significantly less severe pathology compared to Dmd null (e.g., mdx52) mouse.

In certain embodiments, muscles from the mouse have moderately increased levels of inflammatory/necrotic foci, collagen deposition (e.g., +1.4-fold or more) and/or trends of sarcolemmal damage as measured by the intracellular presence of IgM.

In certain embodiments, the subject bmx mouse has increased relative levels of inflammatory transcripts selected from Tlr7, Il1b, Ccl2, Tnf and/or Irf1, such as Ccl2 and/or Il1b, compared to wt mouse.

In certain embodiments, the subject bmx mouse has increased dystrophin-targeting miRNAs (DTMs) and/or inflammatory miRNAs regulated by the inflammatory transcription factor NF-κB, compared to wt mouse.

In certain embodiments, muscles from the mouse show increased expression of NF-kB-driven inflammatory genes and miRNAs, and genes indicative of active fibrosis.

In certain embodiments, the subject bmx mouse has increased expression of fibrosis-associated genes compared to wt mouse, such as Col1a1, Col3a1, and/or Tnc.

In certain embodiments, the subject bmx mouse has increased collagen deposition in quadriceps or increased IgM-positive myofibres compared to wt mouse.

In certain embodiments, serum from the mouse shows increased levels of creatine kinase (CK) compared to wt mouse, indicative of muscle damage.

In certain embodiments, the mouse shows signs of cardiomyopathy or decreased heart function compared to wt mouse, indicative of heart damage.

In certain embodiments, the mouse is homozygous/hemizygous.

In certain embodiments, the mouse is a male.

In certain embodiments, the mouse is a female.

In certain embodiments, the mouse is aged/aging (e.g., at least 3 months, 6 months, 9 months, 12 months, 15 months, 18 months, 21 months, 24 months, 27 months, or 30 months).

In some embodiments, the BMD mice/the subject bmx model of the invention mimics any one or more of the disease phenotypes of the human BMD patients described above.

The data presented herein establish that the bmx mouse model of the invention as a novel model of BMD.

Another aspect of the invention provides a muscle tissue of any one of the mouse of the invention.

In certain embodiments, the muscle tissue is a skeletal muscle tissue.

In certain embodiments, the muscle tissue is a cardiac muscle tissue.

In certain embodiments, the muscle tissue is a smooth muscle tissue.

Another aspect of the invention provides a muscle cell of/from any one of the mouse of the invention.

In certain embodiments, the muscle cell is a primary cell.

In certain embodiments, the muscle cell is a progeny of a cultured cell.

In a related aspect, the invention provide a method of using the bmx mouse to gain new insights into BMD disease pathology, to model dystrophin restoration therapies in DMD, and to facilitate the development of BMD therapeutics as well as DMD co-therapeutics.

Another aspect of the invention provides a method of characterizing BMD disease progression, the method comprising recording, analyzing, and/or characterizing at least one phenotype related to muscular dystrophy in the mouse of the invention, the muscle tissue of the invention, and/or the muscle cell of the invention.

In certain embodiments, the method further comprises applying a candidate therapy to the mouse, the muscle tissue, and/or the muscle cell.

In certain embodiments, the candidate therapy is a small molecule compound, a biologic macromolecule (e.g., antisense polynucleotide), or a viral vector (e.g., a recombinant AAV expressing a gene effective to treat muscular dystrophy such as DMD and/or BMD).

It should be understood that any one embodiment of the invention can be combined with one or more other embodiments of the invention described herein, including any embodiments described only in the examples or claims.

### EXAMPLES

The Examples described herein are for illustrative purpose only, and are not limiting in any respect.

### Example 1 Mouse Model of BMD—Bmx Mice

CRISPR/Cas9 technology was use to generate bmx (Becker muscular dystrophy, X-linked) mice, which express an in-frame ˜40 000 BP deletion of exons 45-47 in the murine Dmd gene, reproducing the most common BMD patient mutation. Further, muscle pathogenesis was characterized using molecular and histological techniques, and then skeletal muscle and cardiac function were tested using muscle function assays and echocardiography.

Results: Overall, bmx mice present with significant muscle weakness and heart dysfunction versus wild-type (WT) mice, despite a substantial improvement in pathology over dystrophin-null mdx52 mice. bmx mice show impaired motor function in grip strength (˜39%, P<0.0001), wire hang (P=0.0025), and in vivo as well as ex vivo force assays. In aged bmx, echocardiography reveals decreased heart function through reduced fractional shortening (˜25%, P=0.0036). Additionally, muscle-specific serum CK is increased >60-fold (P<0.0001), indicating increased muscle damage. Histologically, bmx muscles display increased myofibre size variability (minimal Feret's diameter: P=0.0017) and centrally located nuclei indicating degeneration/regeneration (P<0.0001). bmx muscles also display dystrophic pathology; however, levels of the following parameters are moderate in comparison with mdx52: inflammatory/necrotic foci (P<0.0001), collagen deposition (+1.4-fold, P=0.0217), and sarcolemmal damage measured by intracellular IgM (P=0.0878). Like BMD patients, bmx muscles show reduced dystrophin protein levels (˜20-50% of WT), whereas Dmd transcript levels are unchanged. At the molecular level, bmx muscles express increased levels of inflammatory genes, inflammatory miRNAs and fibrosis genes.

Bmx mice exhibit a phenotype intermediate to WT and mdx mice at the functional, molecular, and histological level. Bmx mice show impaired motor function including reduced forelimb and hindlimb grip strength, (−15%, p=0.0266; −39%, p<0.0001, respectively), wire hang time (p=0.0025), and in vivo isometric torque (−10%, p=0.010). Histologically, bmx muscles have increased myofiber size variability (minimal Feret's diameter: p=0.0017) and centrally located nuclei that indicate of degeneration/regeneration (p<0.0001). Bmx muscles have moderately increased levels of inflammatory/necrotic foci (p=0.0116) and collagen deposition (+1.4-fold, p=0.0083) and showed trends of sarcolemmal damage as measured by the intracellular presence of IgM (p=0.0878). Similar to BMD patients, bmx muscles have reduced dystrophin protein levels (˜20-50% of WT levels) in skeletal and cardiac muscles, while Dmd transcript levels are unchanged. At the molecular levels bmx muscles also show increased expression of NF-kB-driven inflammatory genes and miRNAs as well as genes indicative of active fibrosis.

The bmx mouse of the invention recapitulates the BMD disease phenotypes with histological, molecular and functional deficits. Importantly, it can inform both BMD pathology and DMD dystrophin restoration therapies. This novel model enables further characterization of BMD disease progression, identification of biomarkers, identification of therapeutic targets and new preclinical drug studies aimed at developing therapies for BMD patients.

### Methods

### Mice

All animal studies were conducted according to the NIH Guide for the Care and Use of Laboratory Animals, all national laws and 1964 Declaration of Helsinki standards and amendments and approval of the Institutional Animal Care and Use Committee of Children's National Hospital (CNH).

C57/BL6-mdxA52 mice (mdx52) contain a deletion of exon 52 of the Dmd gene, resulting in absence of full-length dystrophin (Shin'ichi Takeda). These mice are on a C57/BL6 background. Wild-type C57/BL6 (WT) mice were purchased from Jackson Laboratory (Bar Harbor, ME).

### Generation of Bmx Mice+DNA Sequencing

bmdΔ45-47 (bmx) mice were generated on a C57/BL6 background via CRISPR using guide RNAs (gRNAs) to delete exons 45-47 of the murine dystrophin (Dmd) gene. gRNAs were designed to target protospacer adjacent motif (PAM) sequences upstream of Dmd exon 45 and downstream of Dmd exon 47. Subsequent DNA sequencing of pups was performed to verify germline transmission.

### Serum Creatine Kinase

Serum creatine kinase, muscle-type (CKM) was assayed via ELISA according to manufacturer's instructions (Novus Biologicals #NBP2-75306).

### Capillary Gel Electrophoresis (Wes)

Indicated muscles were dissected and frozen in liquid-nitrogen cooled isopentane. 50-100 Sections were then lysed in High SDS buffer Containing 0.02% EDTA (pH 8.0), 0.075% Tris-HCL (pH 6.8), and protein/protease inhibitors. Protein lysates were quantified using DC Bradford Protein Assay (cat #5000111) and analyzed via PerkinElmer EnSpire. Samples were diluted to 4 mg/mL for downstream application. Capillary Western immunoassay (Wes) analysis was performed according to the manufacturer's instructions using a 66-440 kDa Separation Module (ProteinSimple). In each capillary a final protein dilution with a concentration of 0.2 mg/mL was prepped according to protocol and was loaded for dystrophin and vinculin analysis. For dystrophin detection, a rabbit monoclonal anti-dystrophin antibody (ab15277, Abcam, dilution 1:15) was used. In addition, a rabbit monoclonal antibody targeting Vinculin (ab130007, Abcam dilution 1:100) was used to control for muscle content. For both dystrophin and vinculin an anti-rabbit secondary antibody (#042-206, Protein Simple) was used. A representative blot determined by the intensity of chemiluminescence is shown. Compass for SW software was used to obtain chemiluminescence data for dystrophin and vinculin; data is reported as % WT.

### Motor Function Tests

Forelimb and Hindlimb Grip strength. Forelimb and Hindlimb grip strength measurement was assessed using a grip strength meter (Columbus Instruments). Strength was assessed daily for 2 consecutive days using a grip strength meter (Columbus Instruments) according to the Treat NMD SOP (DMD_M.2.2.001). Data was interpreted as maximum daily values for each of five testing days and averaged over the 2 days.

Two-limb wire hang and four-limb grid hang: Both tests were performed in accordance with the Treat NMD protocol (DMD_M.2.1.005). For the two-limb wire hang, a wire hanger was suspended placed ˜35 cm above a cage with soft bedding. Mice were hung using only their forelimbs, however, were allowed to swing and hang with all four limbs if they were able. The hang time was recorded, and 600 seconds was used as a cutoff. For the four-limb grid hang test the same parameters were used (35 cm elevation, 600 second cutoff), but mice instead hung on a handmade box covered in wire mesh (1×1 cm square grid).

In vivo Isometric Torque: To measure in vivo torque production of the anterior crural muscles (TA, extensor digitorum longus (EDL), peroneus tertius, and extensor hallucis longus), mice were anesthetized with 1.5% isoflurane-mixed O₂ and hair was removed from the lower hind limbs, while the foot was attached to the dual-mode lever and maintained at a 90° angle for isometric torque assessment (Aurora Scientific, Aurora, Canada) as detailed in [22]. Isometric muscle contractions were stimulated at 1.0-2.0 mA using Pt—Ir needle electrodes inserted percutaneously adjacent to the peroneal nerve. Peak isometric torque was measured in response to tetanic stimulations at 20, 40, 60, 80, 100, 120, 140, 160, 180, and 200 Hz, providing a 60 s rest period between stimuli.

### Ex Vivo Eccentric Contractions

An eccentric injury protocol was performed in male bmx, mdx52 and WT mice based on previously reported protocols.

### Immunofluorescence

Muscles were harvested from mice and mounted on cork and flash frozen. Frozen unfixed tissue was sectioned (8 μm) onto slides and was stored at −80° C. until ready for use. Frozen slides were thawed for at least 30 minutes at room temperature prior to staining. For all immunofluorescence experiments, except IgM staining, muscle sections were fixed in ice cold acetone for 10 minutes. For anti-IgM immunofluorescence, muscle sections were fixed with 4% paraformaldehyde for 10 minutes at room temperature. Slides were washed with 1×PBST (0.1% Tween 20) 3 times for 10 minutes with gentle agitation. Sections were blocked for 1 hour at room temperature in 1×PBST with 0.1% Triton X-100, 1% BSA, 10% goat serum, and 10% horse serum. Slides were washed 3 times with 1×PBST for 10 minutes with gentle agitation. Primary antibodies were diluted in a solution containing 50% 1×PBST and 50% of the blocking solution (anti-dystrophin 1:150 (Abeam ab154168), anti-laminin-2 1:100 (Sigma-Aldrich Cat. #L0663), anti-mouse IgM-FITC 1:100 (Sigma Aldrich Cat. #F9259), and anti-collagen 1:100 (Abcam ab21286). Slides were incubated in primary antibodies overnight at 4° C. in a humid chamber. The next day, slides were washed 3 times with PBST for 10 minutes each with gentle agitation. Secondary antibodies were diluted in a solution containing 50% 1×PBST and 50% of the blocking solution (goat anti-rabbit 568 1:400 (Thermo Fisher Cat. #A-11036), donkey anti-rat 488 1:400 (Thermo Fisher Cat. #A-21208, goat anti-rat 647 1:400 (Thermo Fisher Cat. #A-21247). Slides were incubated in secondary antibodies for 1.5 hours at room temperature in the dark and then washed 3 times for 10 minutes each with gentile agitation. Coverslips were mounted to slides using Prolong Gold Mounting Medium with DAPI. Slides were imaged using an Olympus VS-120 scanning microscope at 20×.

### BrdU Staining

Mice aged 8-10 weeks were administered water containing 0.8 mg/mL BrdU for 1 week followed by normal water for 1 week. Muscle was then stained for BrdU. Full details can be found in the Supporting Information.

### Muscle Histology Analysis

Cross-sectional area (CSA) and minimum Feret's diameter were determined using the MuscleJ macro for Fiji [23]. The variance coefficient for CSA and minimum Feret's diameter were calculated by dividing the standard deviation by the average and multiplying by 1000. Centrally nucleated fibers were counted manually. Total muscle section area was determined using ImageJ. The total number of myofibers was normalized to the total muscle section area. To quantify muscle damage, the percentage of IgM-positive myofibers was calculated.

### Gene Expression

qRT-PCR of miRNAs and mRNAs was performed.

### Masson Trichrome Staining

Trichrome staining reagents were purchased from Abcam (Cat. #ab150686) and staining was performed following the Treat-NMD SOP “Histopathology in Masson Trichrome stained muscle sections.” Briefly, frozen muscle sections were thawed for 30 minutes at room temperature, fixed in 4% paraformaldehyde for 1 hr at room temperature, and re-fixed in Bouin's solution overnight at room temperature. Slides were washed for 1-2 minutes in running tap water and briefly rinsed in deionized water. Nuclei were stained by incubating sections in equal parts of Hematoxylin Solution A and Solution B for 5 minutes at room temperature followed by rinsing under warm running tap water for 10 minutes and 1 minute in deionized water. Cytoplasm was stained by incubating sections in Biebrich Scarlet-Acid Fuchsin Solution for 5 minutes. Slides were washed 3 times for 1 minute with deionized water and then incubated in phosphotungstic/phosphomolybdic acid solution for 10 minutes. Collagen was stained by incubating slides in Aniline Blue Solution for 5 minutes followed by 3 washes with deionized water for 1 minute. Sections were incubated with 1% Glacial Acetic Acid for 2 minutes followed by 2 washes with deionized water for 1 minute. Sections were dehydrated successively in 70%, 90%, and 100% ethanol for 3 minutes each and then in xylene for 5 minutes. Coverslips were mounted with permount. Slides were imaged using an Olympus VS-120 scanning microscope at 20×.

To analyze images for fibrosis, the Colour Deconvolution 2 plugin for ImageJ was used to separate the dyes [24]. The image containing Aniline blue staining was thresholded, the muscle section was outlined, and the percent of the area of Aniline blue staining (fibrotic area) was determined using the measure tool in ImageJ.

### Hematoxylin and Eosin Staining

Frozen muscle sections were thawed for 30 minutes at room temperature, fixed in 100% methanol for 5 minutes, and washed with deionized water for 30 seconds. Slides were then fixed in hematoxylin for 10 minutes and washed under warm running tap water for 10 minutes. Slides were fixed in eosin for 3 minutes followed by 30-second washes in 50%, 70%, and 90% ethanol and a 1-minute wash in 100% ethanol. Slides were then fixed in xylene and mounted with permount. Slides were imaged using an Olympus VS-120 scanning microscope at 20×.

To analyze necrosis and inflammation, the Colour Deconvolution 2 plugin for ImageJ was used to separate the dyes [24]. The image containing the eosin staining was thresholded, the muscle section was outlined, and the percent of the area without eosin stain was determined using the measure tool in ImageJ. The percentage of area with inflammation and necrosis was calculated by subtracting the percent area without eosin stain from 100.

### Ribonucleic Acid Isolation and Gene Expression Analysis

qRT-PCR of miRNAs and mRNAs was performed as previously reported [25]. ˜50-100 sections (8 μM thickness) of mouse quadriceps muscle or human biopsies were homogenized in 1 mL Trizol (Life Technologies) a TissueRupter II homogenizer (Qiagen), following the manufacturers protocol. miRNAs. Total RNA was converted to cDNA using multiplexed RT primers and High-Capacity cDNA Reverse Transcription Kit (ThermoFisher; Carlsbad, CA). miRNAs were then quantified using individual TaqMan assays on an ABI QuantStudio 7 real time PCR machine (Applied Biosystems). Assay IDs include: miR-146a 000468, miR-146b 001097, miR-223 002295, miR-31 00185, miR-320a 002277, miR-382 000572, miR-374a 000563, miR-142-3p 000464, miR-142-5p 002248, miR-301a 000528, miR324-3p 002509, miR-455-3p 002455, miR-455-5p 001280, miR-497 001346, miR-652 002352, sno202 001232, RNU48 001006.

mRNAs. RNA was isolated from muscle using TRIzol. cDNA was synthesized from 1000 ng RNA using a High-Capacity Reverse Transcription Kit (Thermo Fisher Cat. #4368813). qRT-PCR analysis was performed using TaqMan Fast Advanced Master Mix (Thermo Fisher Cat. #4444557) and TaqMan probes (all Thermo Fisher). miRNAs were then quantified using individual TaqMan assays on an ABI QuantStudio 7 real time PCR machine (Applied Biosystems).

### Statistical Analysis

Statistical analyses were performed using GraphPad Prism v.9.0.0 (GraphPad Software, Inc.). A one-way ANOVA was performed with a Holms-Sidak post-hoc test specifically comparing WT vs. bmx and bmx vs. mdx groups. For all graphs, data are presented as mean±SEM.

### Results

### Generation of a Becker Muscular Dystrophy Mouse Model

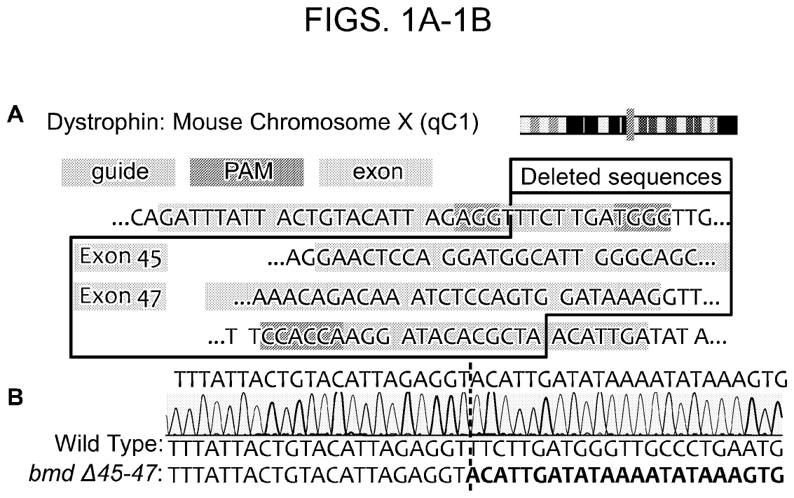

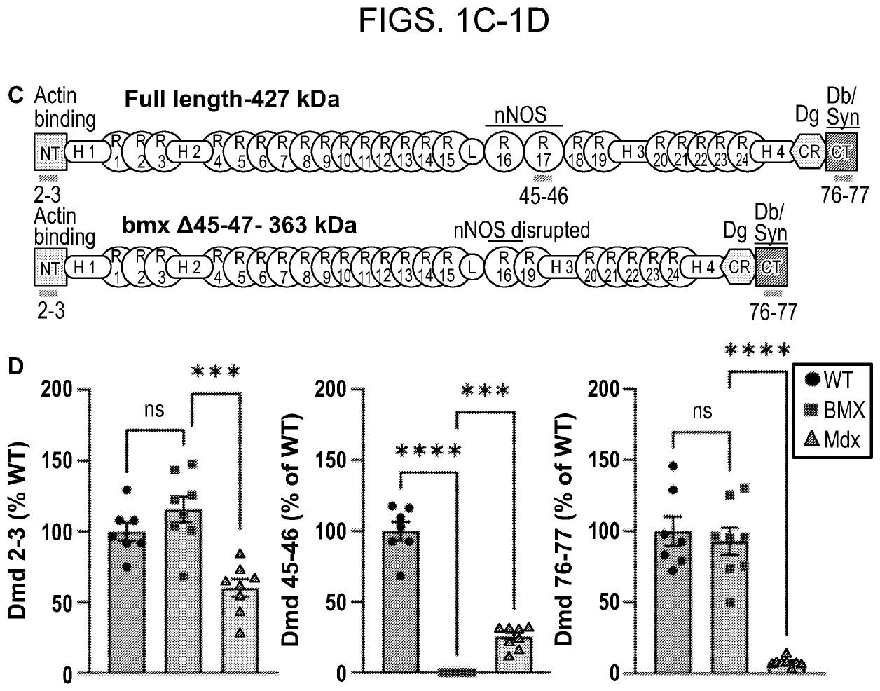

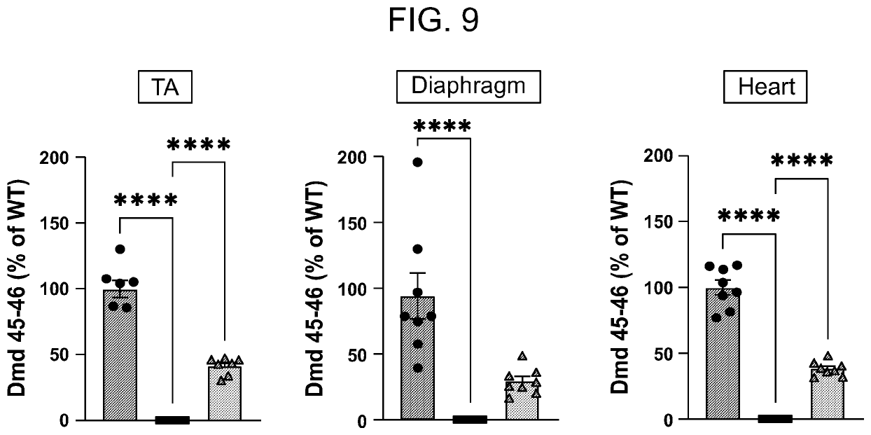

To create bmx mice (for Becker muscular dystrophy, X-linked) that model the most common BMD patient mutation, CRISPR/Cas9 was used to introduce a ˜40,000 bp genomic deletion into the endogenous murine dystrophin (Dmd) gene, excising exons 45-47. Guide RNAs (gRNAs) were designed to target protospacer adjacent motif (PAM) sequences upstream of Dmd exon 45 and downstream of Dmd exon 47 (FIG. 1A). DNA sequencing confirmed genomic deletion of dystrophin exons 45-47 (FIG. 1B), which is predicted to disrupt spectrin-type repeats (STRs) and neuronal nitric oxide synthase (nNOS) binding (FIG. 1C). qRT-PCR was used to validate deletion of exons 45-47 and to quantify overall Dmd transcript levels. Similar expression of Dmd between WT and bmx mice was observed using probes at the 5′ and 3′ ends of the Dmd transcript (exon 2-3 or 76-77), while using a probe for exons Dmd 45-46 confirmed bmx quadriceps (FIG. 1D), TA, heart and diaphragm (FIG. 9) lack this region. Progressively reduced amounts of Dmd mRNA were observed in the 5′-3′ direction in mdx52, consistent with 3′ destabilization as previously reported (FIG. 1D).

### Impaired Motor Function and Reduced Muscle Force in Bmx Mice

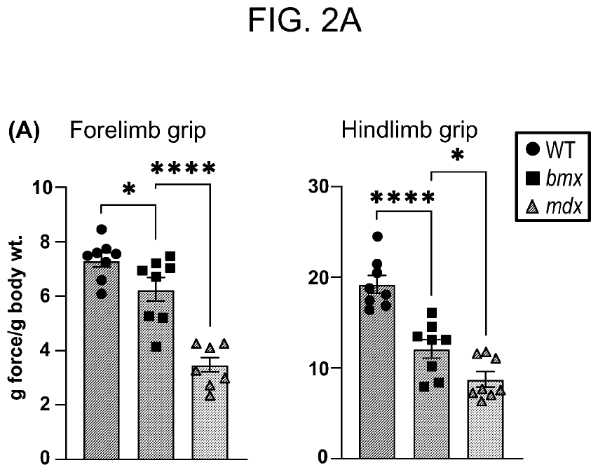

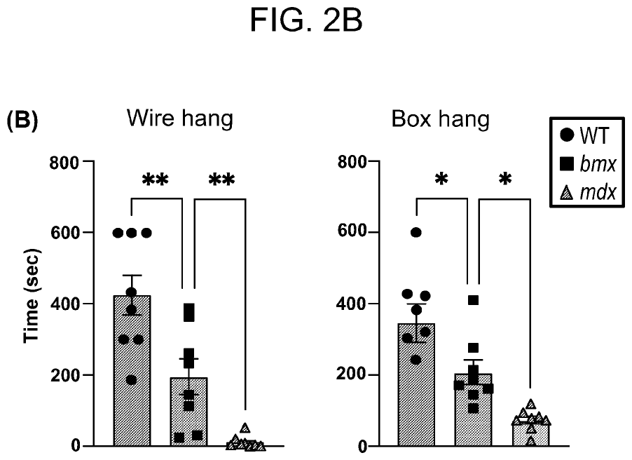

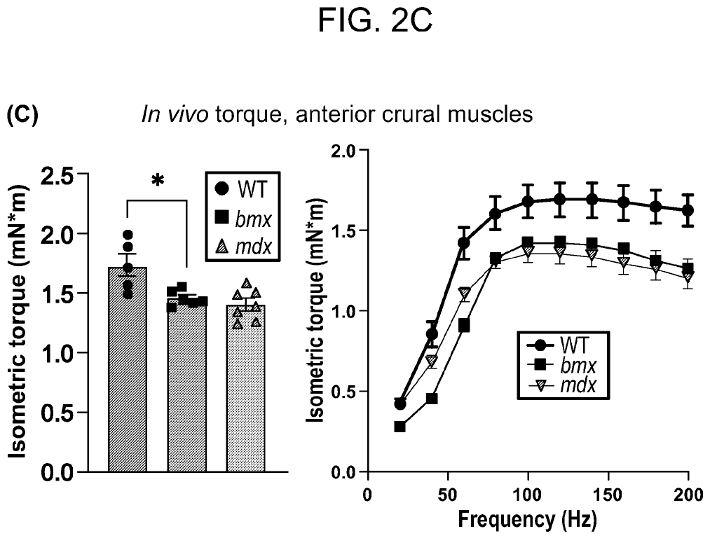

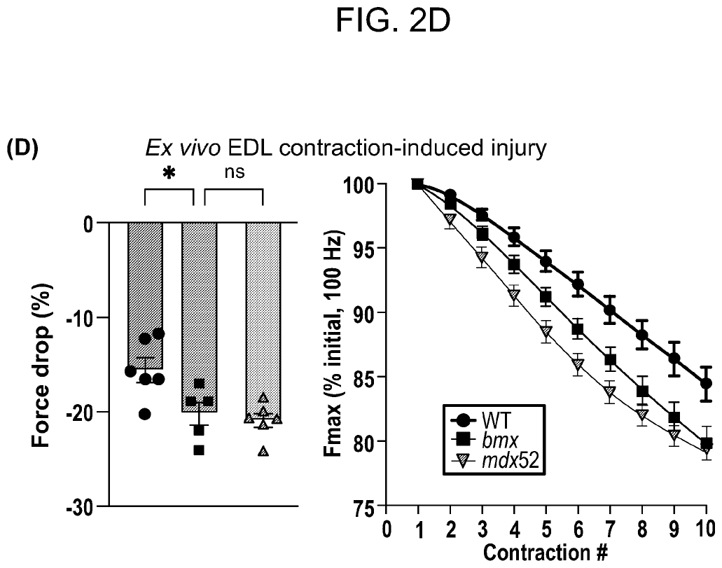

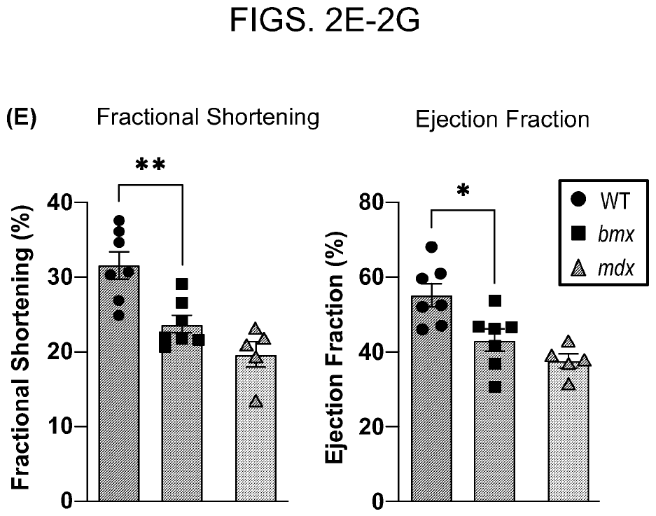

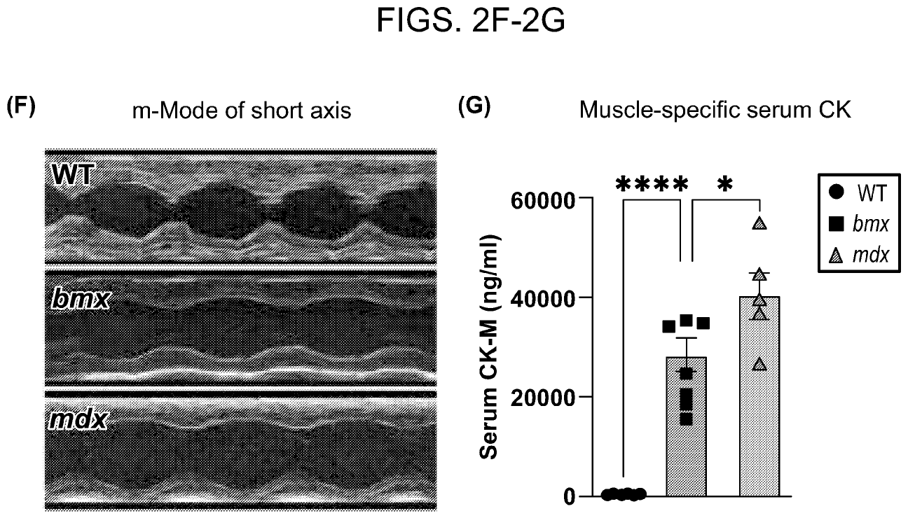

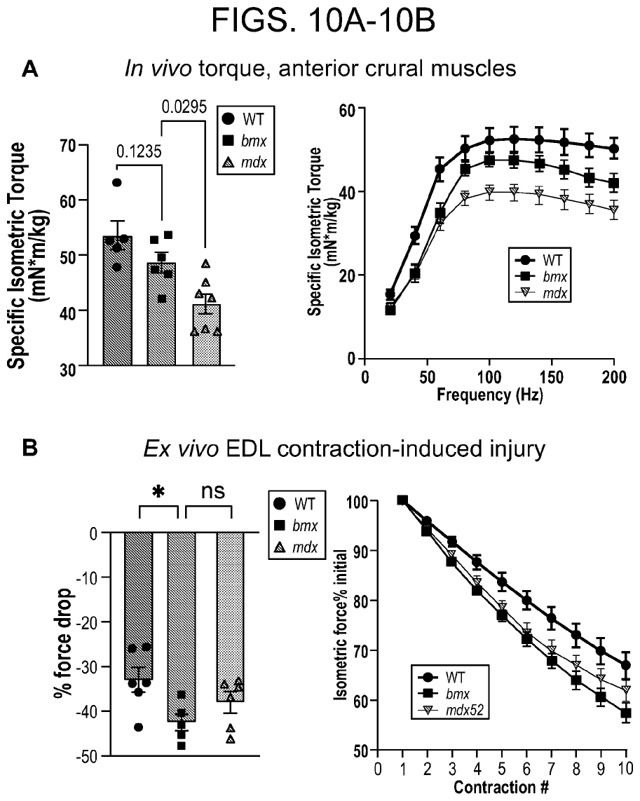

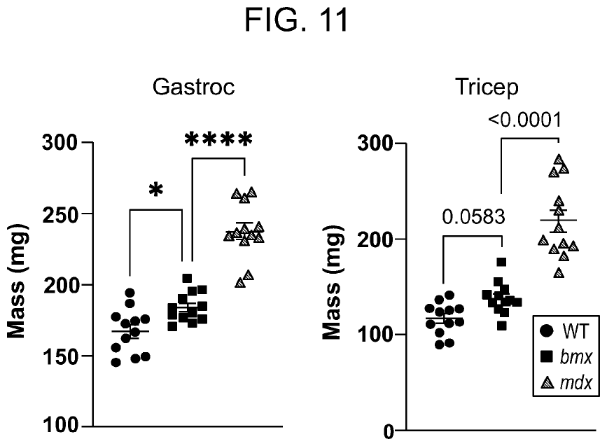

To determine whether bmx mice exhibit functional impairments, phenotyping was performed at 10 weeks (forelimb/hindlimb grip strength), 14 weeks (two-limb wire hang), and 15 weeks (four-limb grid hang). The bmx mice showed reduced grip strength versus WT for forelimb (14.90% decrease, P=0.0465) and hindlimb (36.81% decrease, P<0.0001) (FIG. 2A). Reduced suspension times for bmx were also found in wire hang (˜54.25%, P=0.0087) and box hang (˜39.85%, P=0.0489) (FIG. 2B). Assaying in vivo isometric torque produced in the tibialis anterior (TA), bmx mice showed significantly reduced maximum isometric torque vs. WT mice (1.465 vs. 1.650 mN*m, P=0.0110), though there was no significant improvement in bmx versus mdx52 (FIG. 2C, left). Additionally, the deficit in isometric torque was observed at all frequencies for bmx (FIG. 2C, right). In vivo specific isometric torque showed modest reductions in maximal values for bmx versus WT (48.74 vs. 53.63 mN*m/kg, P=0.1235) and were significantly elevated versus mdx52 (P=0.0295) (FIG. 10A).

Contractile performance and resistance to eccentric injury were examined in bmx mice. Ten eccentric contractions of WT EDL muscle resulted in a 15.53% drop in ex vivo peak force. In contrast, this protocol reduced peak force by 20.16% in bmx EDL muscles (P=0.0239 bmx vs. WT) and 20.86% in mdx EDL (FIG. 2D). This injury protocol also resulted in a 42.49% drop in isometric force in bmx EDL (P=0.0307) vs. 33% isometric force drop in WT and a 38% drop in mdx EDL (FIG. 10B). BMD patients have muscle weakness in proximal and distal limbs leading to motor impairments [7].

Specific isometric torque was determined by normalizing to isometric torque to body weight. By this measure bmx mice showed modest reductions (53.63 vs. 48.74 mN*m/kg, p=0.0699). Specific torque was also reduced at all frequencies and showed measurements intermediate to WT and mdx (FIG. 2D).

### Aged Bmx Mice Develop Heart Dysfunction and Increased Serum CK

Heart function was assessed via echocardiography in 18-month-old WT, bmx and mdx52 mice. This age was chosen because reduced heart function is observed in mdx52 mice starting around 12 months of age. Thus, comparable heart function declines in bmx should therefore be present by 12-18 months of age. The bmx mice showed significant declines in heart function measures (FIGS. 2E & 2F), including fractional shortening and ejection fraction (˜25.07%, P<0.0036, ˜27.67% P<0.0131 vs. WT).

Serum creatine kinase levels were also assessed in aged mice. The bmx mice showed an ˜62-fold (P<0.0001) increase in serum creatine kinase, muscle-type (CKM) over WT (FIG. 2G). The bmx serum CKM levels (28,447 ng/mL) were intermediate to WT (457 ng/mL) and mdx (40,514 ng/mL), and bmx CKM was significantly lower than mdx (P=0.0217). These data indicate aged bmx mice have reduced heart function and increased serum CK, consistent with human disease.

### Bmx Mice Undergo Muscle Hyperplasia and Hypertrophy

In mdx mice, limb muscles undergo dramatic necrosis at 3 to 4 weeks of age followed by robust regeneration accompanied by an increased number of muscle fibers, as well as muscle hypertrophy. Muscle hypertrophy causes mdx mice to be larger than normal between about 10 and 40 weeks of age, after which they lose weight in concert with a loss of muscle mass (loss of muscle mass typically occurs in older mdx mice (12-18 months)). Because of the massive regeneration early on, and because of the increase of larger regenerated fibers as a function of age, the coincidence of both large and small fibers suggests that both hyperplasia and true hypertrophy contribute to mdx muscle hypertrophy; this models what is seen in DMD patients. Similarly, BMD patients show muscle hypertrophy, which MRI show is a mix of both pseudohypertrophy (increased fibrofatty replacement) and true hypertrophy.

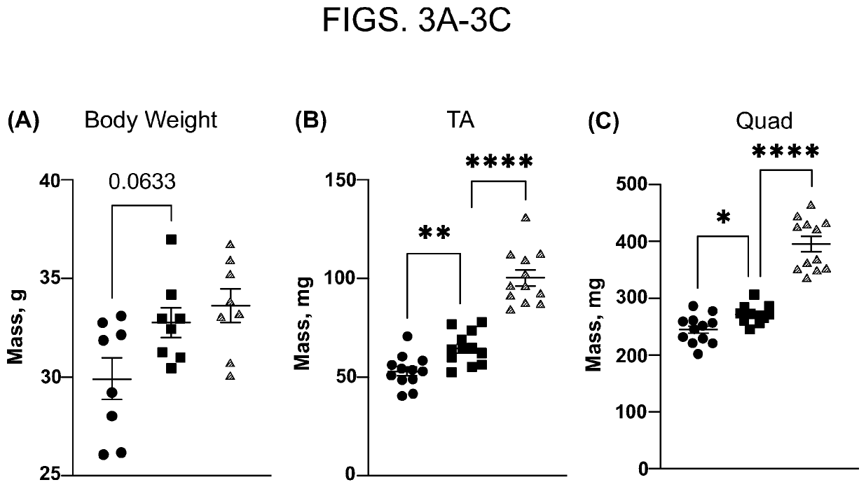

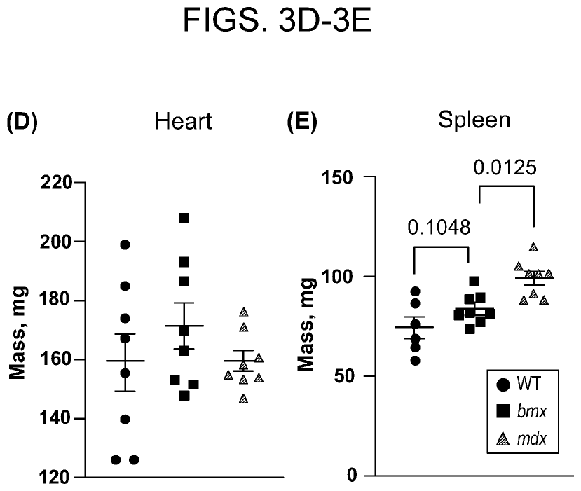

Examining body and muscle mass at 5 months, bmx showed a 9.57% increase (P=0.0633) in body weight compared with WT mice (FIG. 3A). Increased muscle mass was present in every skeletal muscle examined: TA (18.16% increase, P=0.0096), quadriceps (9.75% increase, P=0.0456), gastrocnemius (9.135% increase, P=0.0143), and triceps (17.95% increase, P=0.0583) (FIGS. 3B, 3C, and 11). No significant difference was seen in heart mass (P=0.4288) versus WT and bmx mice (FIG. 3D).

As mdx52 spleens are enlarged due to increased systemic inflammation, spleen mass was also examined. A moderate increase in bmx spleen mass was observed, which did not reach significance (12.45% increase, P=0.1048) (FIG. 3E), but may suggest increased circulating inflammation in bmx.

Biopsies from BMD patient muscles show an increased variation in myofiber size and an increase in centrally nucleated fibers which is indicative of increased both increased muscle regeneration and asynchronous regeneration, and the presence of larger regenerating fibers.

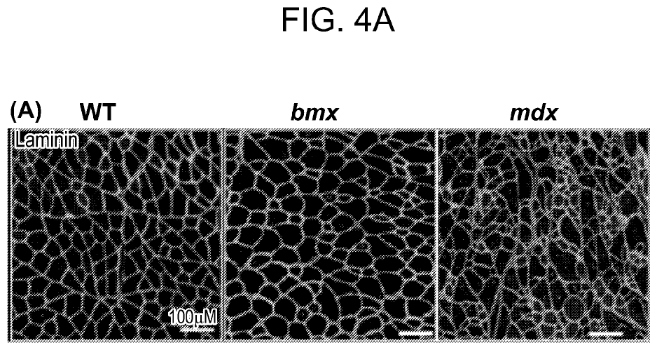

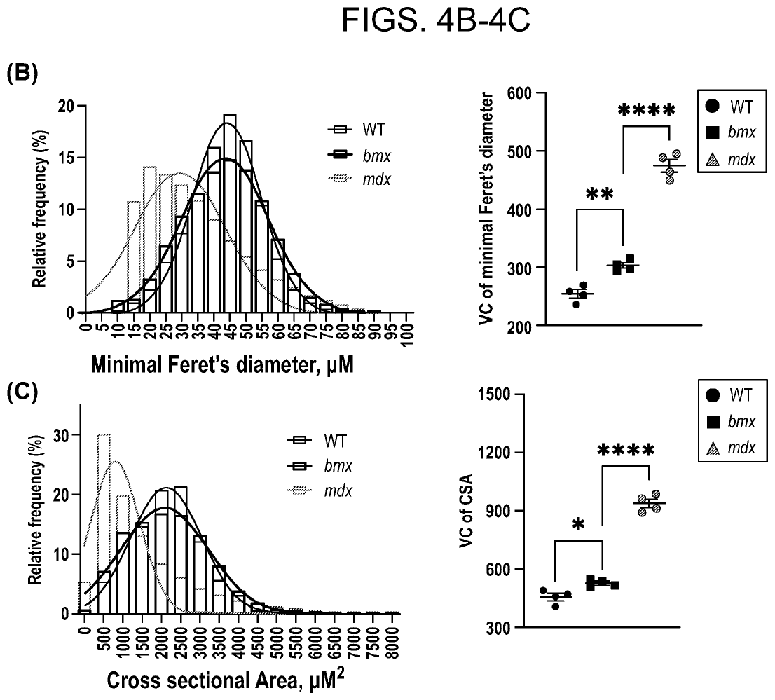

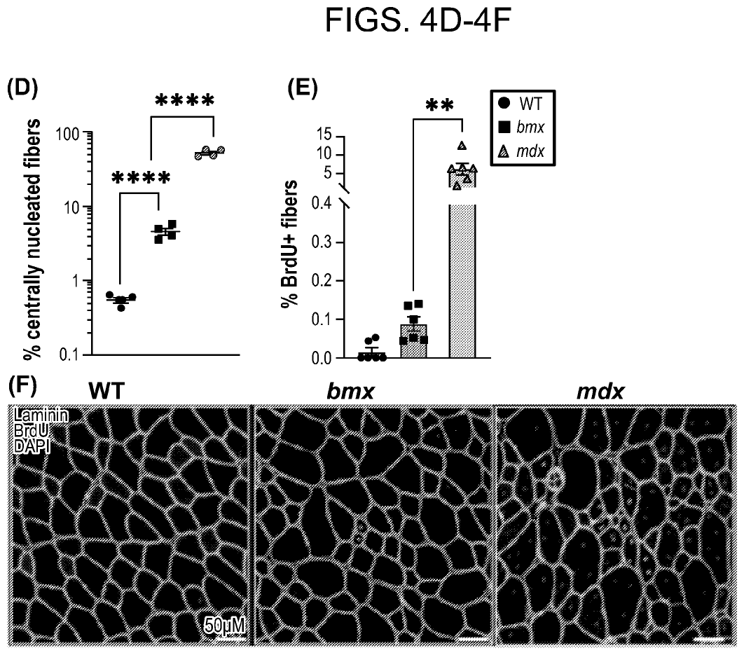

BMD muscle biopsies show increased myofibre size variability and centrally nucleated fibres, indicative of increased and asynchronous muscle regeneration. To examine bmx muscle architecture, gastrocnemius muscle sections were immunostained for laminin-α2. Visually, fibre size variability and some centrally localized nuclei in bmx muscles were observed, indicating pathology (FIG. 4A). Muscle fibre measurements including minimal Feret's diameter and myofibre cross-sectional area (CSA) were then determined. Whereas mdx52 showed a shift towards smaller, regenerating fibres, with some intermittent large fibres, bmx mice showed a greater number of both smaller and larger myofibres versus WT, as indicated by histograms plotting minimal Feret's diameter and CSA measurements (FIGS. 4B & 4C).

Differences in fibre size variation were additionally determined by calculating variance coefficients (VCs). VCs showed significant differences between fibre size variability for bmx versus WT, for minimal Feret's diameter (+18.91%, P=0.0017) and CSA (+14.96%, P=0.0182) (FIGS. 4B & 4C). bmx also had significantly more centrally nucleated fibres versus WT (4.573% vs. 0.5594%, P=0.0002) (FIG. 4D).

Next, the thymidine analogue bromodeoxyuridine (BrdU) was used to label recently generated myofibres, as BrdU incorporates into newly synthesized DNA and therefore labels newly “born” myofibres. Mice were given BrdU for 7 days at 19 weeks of age; BrdU+ centralized nuclei-containing myofibres were counted as an indicator of myoblasts that had proliferated and fused into myofibres during labelling. Approximately five percent of mdx52 myofibres were BrdU+ after 1 week of labelling (FIGS. 4E & 4F). Although there were very few BrdU+ fibres in WT TAs, bmx showed a significant increase in the percentage of actively regenerating muscle fibres, which was intermediate to WT and mdx52 (0.1%, P=0.0058; FIGS. 4E & 4F). These data show that bmx mice have increased regeneration and myofibre hypertrophy versus WT muscles while also having significantly less severe pathology versus mdx52.

### Reduction of Dystrophin Protein in Bmx Mice

BMD patients with deletion of DMD exons 45-47 have decreased and variable dystrophin protein levels, whereas mRNA levels are unchanged. To determine if bmx mice recapitulate this phenotype, qRT-PCR was performed using a probe against the Dmd 76-77 exon junction. This junction was chosen as previous work shows that a 3-5′ destabilization of Dmd mRNA occurs in mdx52 and DMD muscle. Thus, a 3′-specific probe was more likely to show apparent differences in transcript abundance and stability.

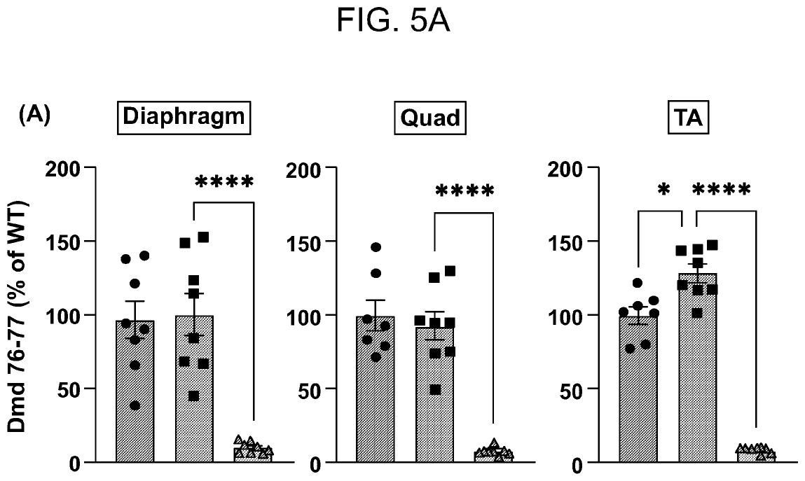

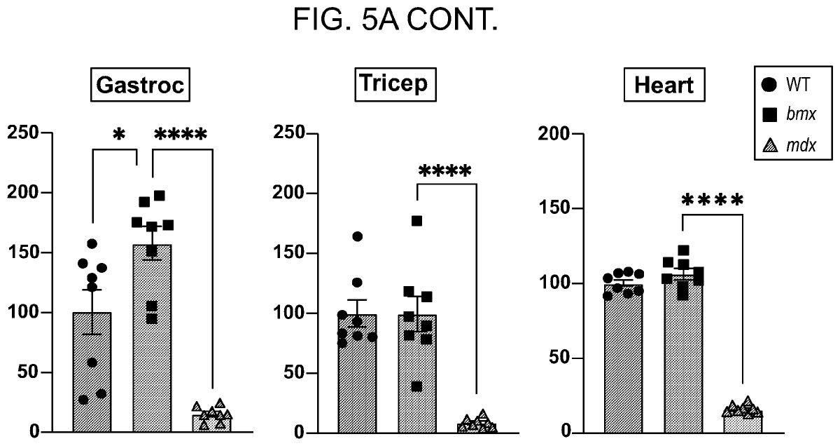

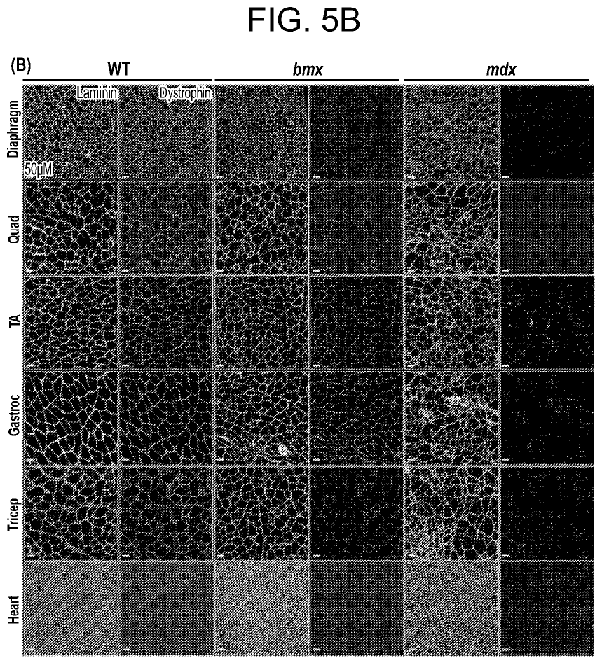

In all muscles measured (diaphragm, quad, TA, gastroc, triceps, heart), Dmd transcript levels were not decreased in bmx, whereas expectedly, Dmd was reduced in mdx52 muscles (FIG. 5A). Additionally, in both TA and gastrocnemius muscles, bmx showed higher levels of Dmd mRNA than WT (TA+28.85%, P=0.0005, gastroc+57.54%, P=0.0061). This shows that there is not a Dmd gene expression deficit in bmx, and conversely, in some muscles, Dmd transcript levels are more abundant than in WT.

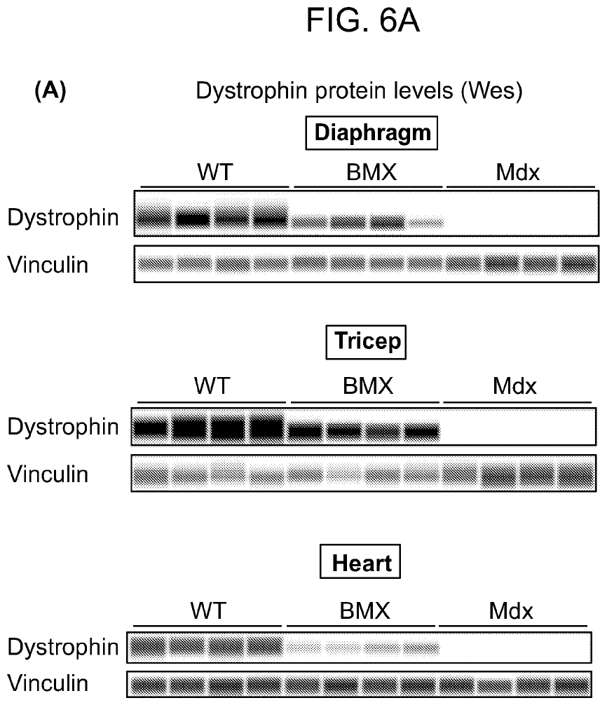

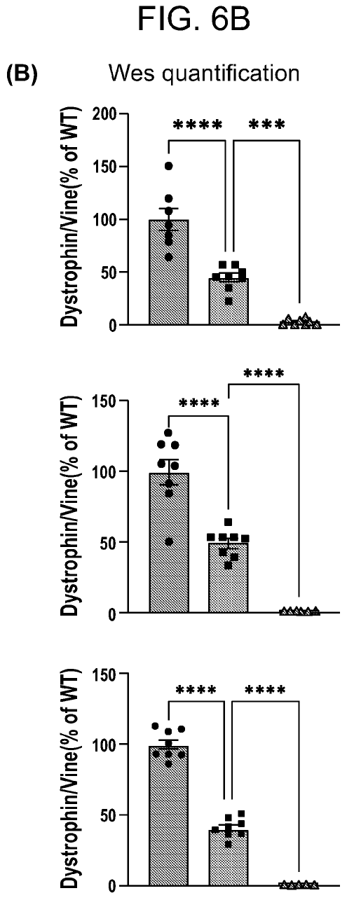

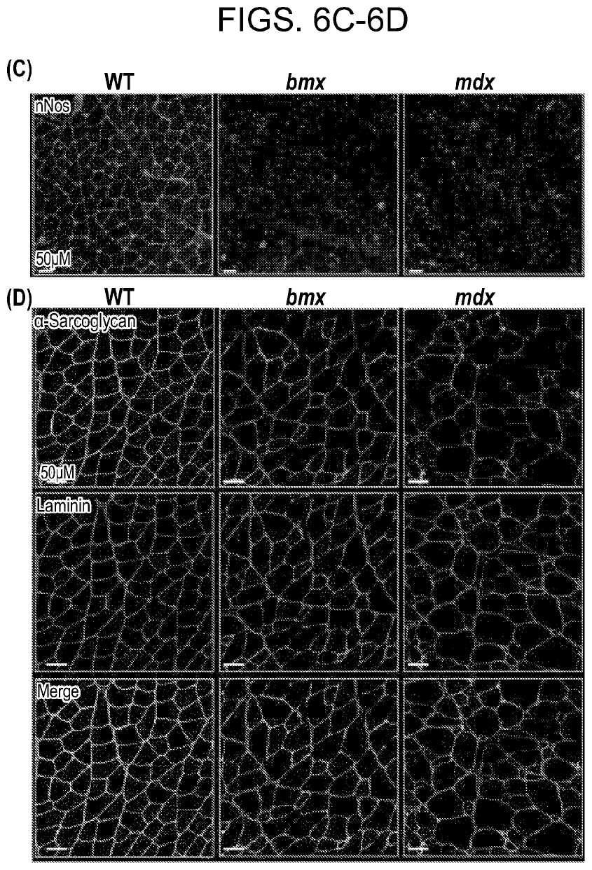

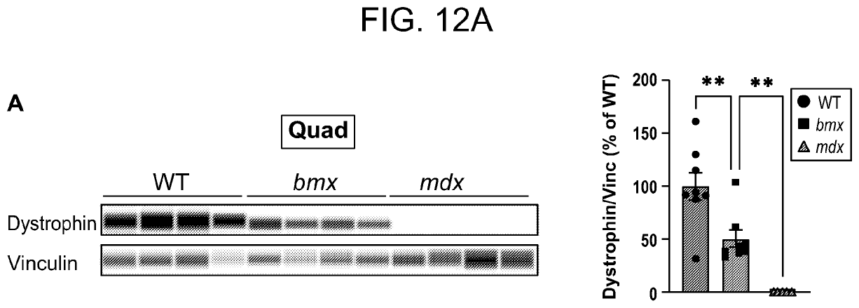

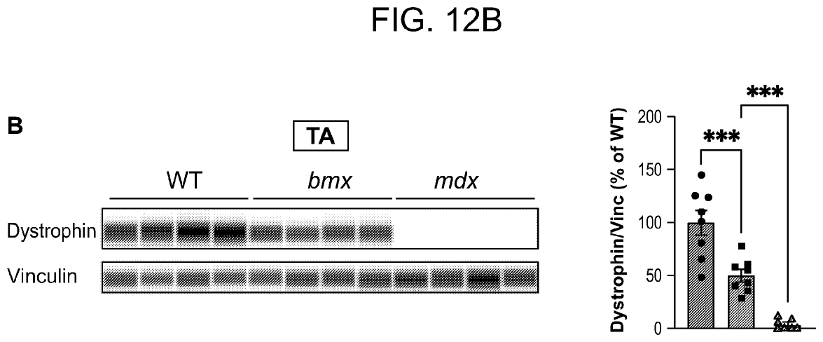

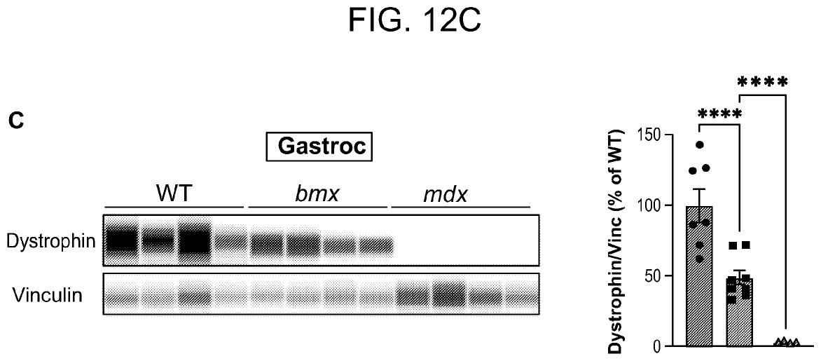

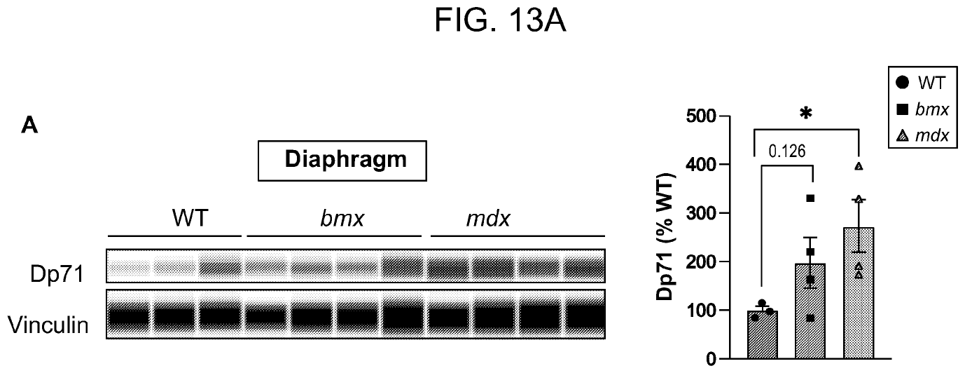

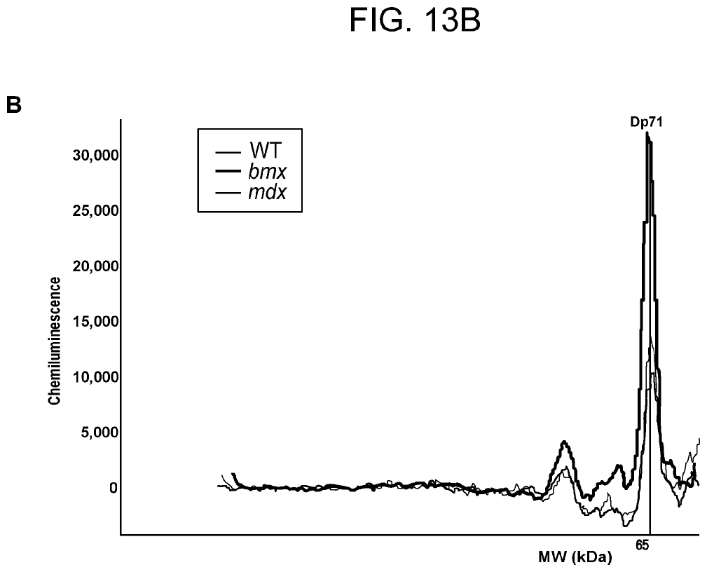

Next, dystrophin protein levels were assessed via immunofluorescence. Visual reductions were observed in bmx skeletal and cardiac muscles versus WT (FIG. 5B). As Capillary Western immunoassays (Wes) has proven to be highly sensitive, reproducible and quantitative over a large dynamic range for dystrophin quantification, Wes was used to prove that bmx skeletal and heart muscles showed, on average, ˜50% less dystrophin than WT (diaphragm, P=0.0002; triceps, P<0.0001, heart, P=0.0001; quadriceps, P=0.0064; TA, P=0.0016; gastroc, P=0.0010) (FIGS. 6A, 6B, and 12A-12C). In the diaphragm, the shorter, C-terminal, ubiquitously expressed dystrophin isoform Dp71 was also detected. Consistent with previous reports, a ˜2.5-fold increase in Dp71 expression was observed in mdx52 diaphragms (P=0.0445; FIGS. 13A and 13B). bmx showed slightly elevated levels of Dp71 (˜2-fold); however, this did not reach statistical significance (P=0.126; FIGS. 13A and 13B).

Next, localization of nNOS and of the dystrophin-associated protein (DAP) α-sarcoglycan were determined. Consistent with loss of dystrophin STR R17 (encoded by exon 45), which is necessary for nNOS binding, bmx muscles showed complete loss of nNOS localization at the sarcolemma as did dystrophin-null mdx52 muscles (FIG. 6C). α-sarcoglycan localization, based on immunofluorescence, showed both reduced amounts and reduced colocalization at myofibre membranes in both bmx and mdx52 muscle (FIG. 6D). Collectively, these data show bmx exhibit reduced association between dystrophin and DAPs at the sarcolemma; this is likely directly linked with the internal dystrophin truncation (nNOS) and reduced dystrophin levels (α-sarcoglycan) in bmx muscle.

Bmx Muscles Express Higher Levels of Inflammatory Genes and miRNAs

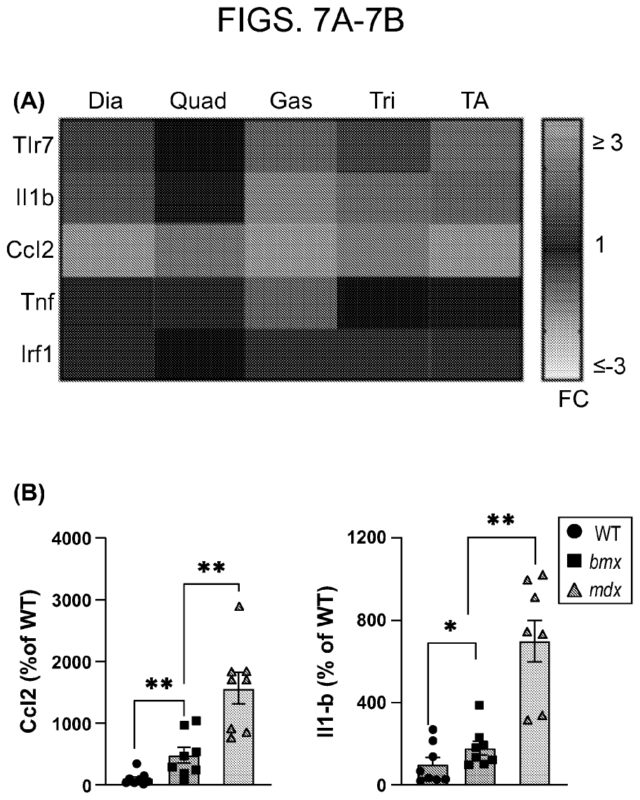

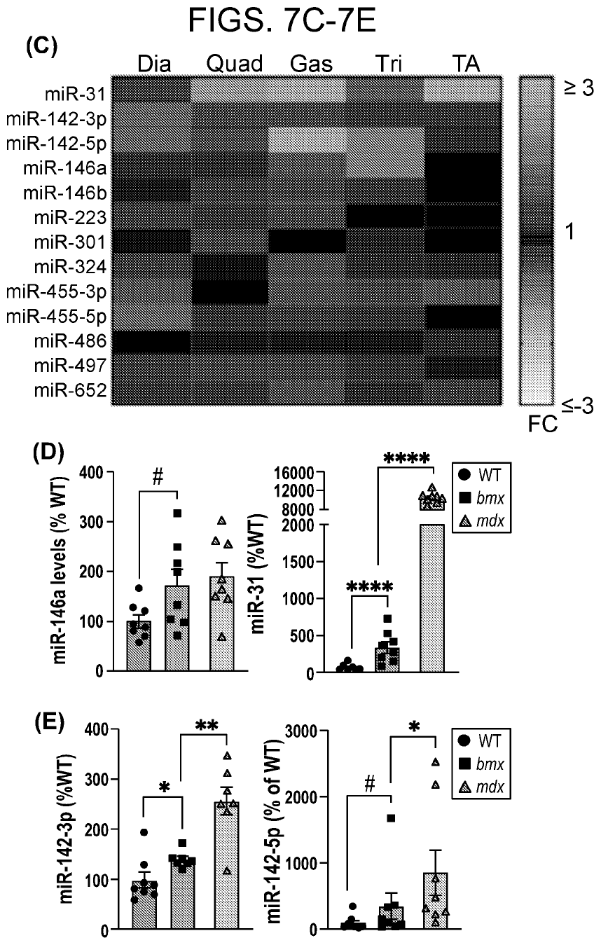

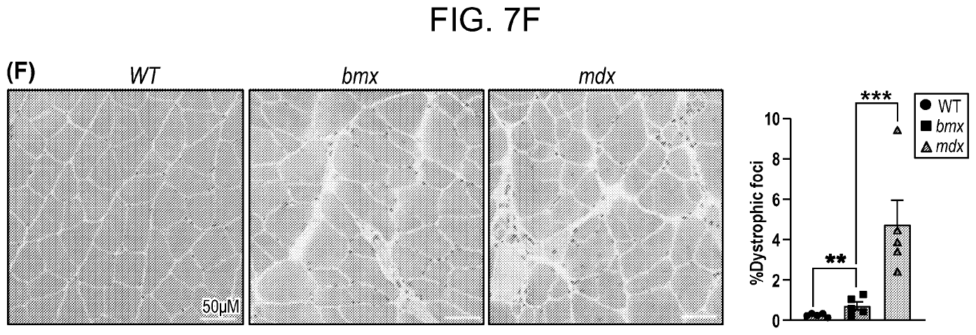

Levels of inflammation-induced genes were also examined, using a pre-determined inflammatory panel consisting of Tlr7, Il1b, Ccl2, Tnf and Irf1. Relative levels of inflammatory transcripts for all bmx muscles were determined versus WT, and were plotted as a heat map (FIG. 7A), which demonstrated that the gastroc muscle has the highest levels of inflammatory transcripts, and Ccl2 is the most highly upregulated transcript across all bmx muscles.

Individual transcript levels for WT, bmx and mdx52 gastrocs were also plotted, which demonstrated significant increases in Ccl2 (+375%, P=0.0012) and Il1b (+80%, P=0.0140) (FIG. 7B). Also, miRNA panels—dystrophintargeting miRNAs (DTMs) and inflammatory miRNAs—both regulated by the inflammatory transcription factor NF-κB, were used to generate a heat map, showing levels of inflammatory-driven miRNAs in bmx versus WT. The heat map revealed that the gastroc expresses the highest levels of DTMs and inflammatory miRNAs (FIG. 7C). Plotting individual miRNA expression levels in gastroc demonstrated increased DTMs (miR-146a, 72% increase, P=0.0928; miR-31, 265% increase, P<0.0001) and inflammatory miRNAs (miR-142-3p, 33.81% increase, P=0.0183; miR-142-5p, 255.7% increase, P=0.0583; FIGS. 7D & 7E).

BMD muscle biopsies show immune cell infiltration and myofibre necrosis. As bmx gastroc muscles were most affected at the molecular level, sections were stained with haematoxylin and eosin (H&E) to quantify inflammation and necrosis (dystrophic foci). H&E staining visually demonstrated pathology in bmx, and quantification showed a 29% increase (P=0.0021) in dystrophic foci as compared with WT (FIG. 7F).

### Fibrotic Gene Expression and Staining are Increased in Bmx

DMD muscles show high levels of fibrosis, and BMD patient muscles show variable fibrosis which inversely correlates with functional measures. In mdx52, fibrosis is apparent in the diaphragm by 8-10 months of age, and in other skeletal muscles by 20 months. Preceding obvious fibrosis, elevated levels of fibrosis-associated genes and increased deposition of perimysial and endomysial collagen are observed.

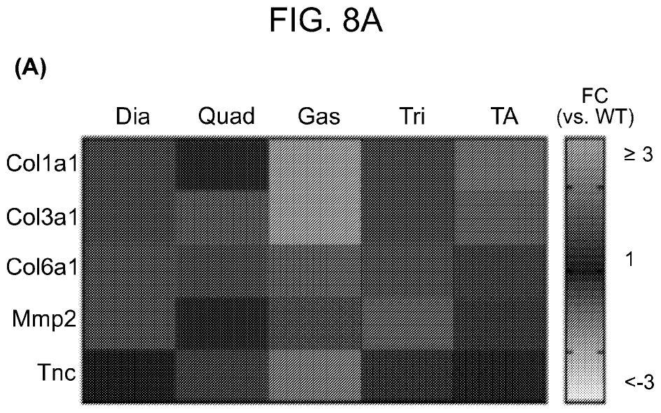

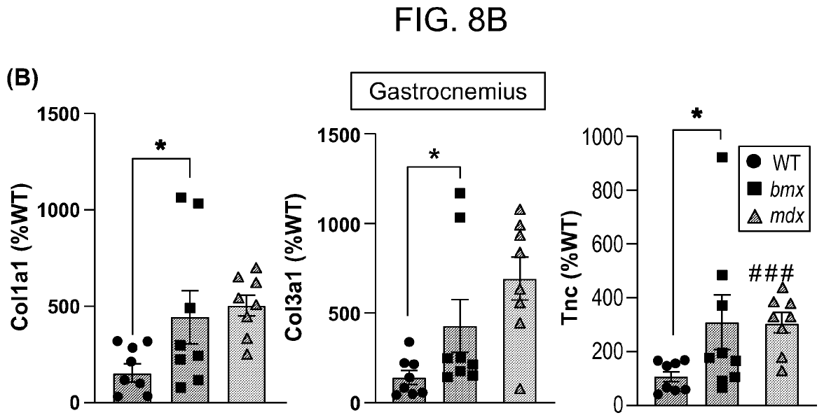

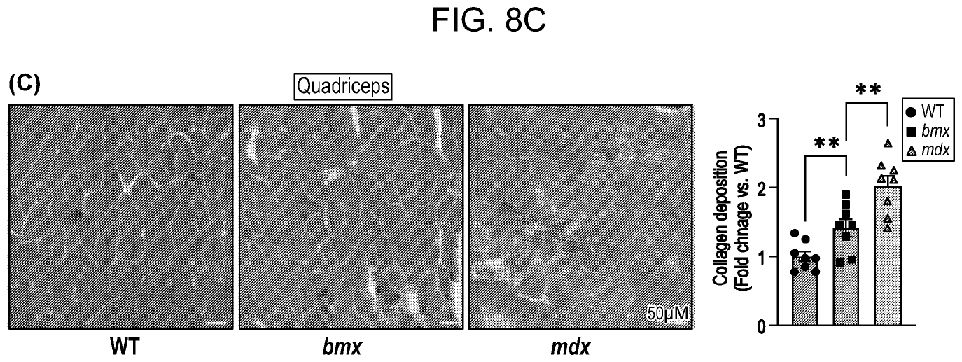

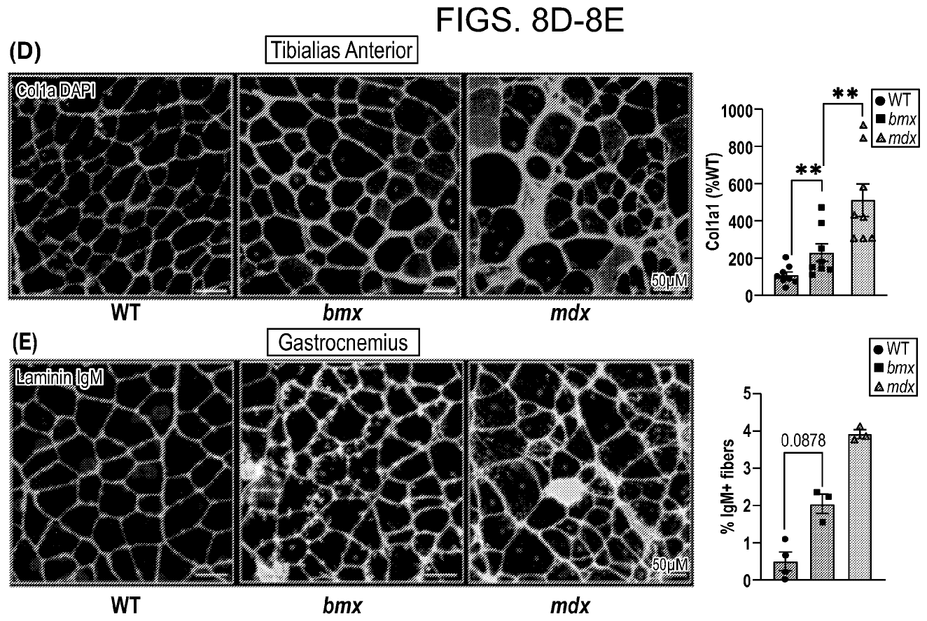

A panel of genes indicative of fibrosis signaling, including Col1a1, Col3a1, Col6a1, Mmp2 and Tnc36,37 were examined, and their relative levels in bmx (vs. WT) were plotted as a heat map for all muscles examined (FIG. 8A). The bmx gastroc showed the highest upregulation of fibrosis-associated genes (FIGS. 8A & 8B). Representative graphs of a few fibrosis-associated transcripts show significant increases in Col1a1 (+188%, P=0.0288), Col3a1 (+207%, P=0.0452), and Tnc (+188%, P=0.0273) in bmx gastroc (FIG. 8B), as well as Col3a1 and Tnc in the TA (data not shown). Masson's trichrome staining showed increased collagen deposition in bmx versus WT quadriceps (P=0.0083; FIG. 8C), and Collagen 1a immunofluorescence showed visually increased collagen around the sarcolemma in bmx TA (FIG. 8D). qPCR supported this observation (FIG. 8D).

Increased fibrosis is often accompanied by increased muscle damage. To assess muscle damage in bmx mice, quadriceps muscles were immunostained with an anti-IgM antibody, which preferentially accumulates in damaged myofibers. bmx mice had a visual increase in IgM-positive myofibres compared with WT mice (FIG. 8E; P=0.0878), indicative that even with ˜50% dystrophin protein, some myofiber damage persists.

### REFERENCES

1. Koenig et al., Am J Hum Genet 1989; 45:498-506.
2. Hoffman et al., N Engl J Med 1988; 318:1363-1368.
3. Hoffman et al., Cell 1987; 51:919-928.
4. Ringel et al., Arch Neurol 1977; 34: 408-416.
5. Comi et al., Brain 1994; 117:1-14.
6. Bradley et al., Muscle Nerve 1978; 1:111-132.
7. Bushby et al., I Natural History J Neurol 1993; 240:98-104.
8. Emery and Skinner, Clin Genet 1976; 10:189-201.
9. Finsterer and Stollberger, Cardiology 2003; 99:1-19.
10. Finsterer and Stollberger, Can J Cardiol 2008; 24:786-792.
11. van den Bergen et al., J Neurol Neurosurg Psychiatry 2014; 85:747-753.
12. Beggs et al., Am J Hum Genet 1991; 49:54-67.
13. Hoffman et al., Neurology 1989; 39:1011-1017.
14. Kesari et al., Hum Mutat 2008; 29:728-737.
15. Fiorillo et al., Cell Rep 2015; 12:1678-1690.
16. Flanigan, Neurol Clin 2014; 32:671-688, viii.
17. Bulfield et al., Proc Natl Acad Sci U.S.A 1984; 81:1189-1192.
18. Iyer et al., Muscle Nerve 2020; 62:757-761.
19. Nakamura et al., Sci Rep 2014; 4:5635.
20. Chemello et al., Proc Natl Acad Sci USA 2020; 117:29691-29701.
21. Araki et al., Biochem Biophys Res Commun 1997; 238:492-497.
22. Spitali et al., FASEB J 2013; 27:4909-4916.
23. Coulton et al., Neuropathol Appl Neurobiol 1988; 14:53-70.
24. Petrof et al., Proc Natl Acad Sci USA 1993; 90:3710-3714.
25. Monforte et al., J Neurol Sci 2014; 347:301-304.
26. Dadgar et al., J Cell Biol 2014; 207:139-158.
27. Mayeuf-Louchart et al., Skelet Muscle 2018; 8:25.
28. Beekman et al., PLoS ONE 2018; 13:e0195850.
29. Kawaguchi et al., Int J Mol Sci 2018; 19:1546.
30. Molza et al., J Biol Chem 2015; 290:29531-29541.
31. Fiorillo et al., Physiol Genomics 2018; 50:735-745.
32. Markand et al., Neurology 1969; 19:617-633.
33. Ripolone et al., Acta Neuropathol Commun 2022; 10:48.
34. Ardite et al., J Cell Biol 2012; 196:163-175.
35. Pessina et al., Skelet Muscle 2014; 4:7.
36. Heier et al., Life Sci Alliance 2019; 2.
37. Kherif et al., Dev Biol 1999; 205:158-170.
38. Teramoto et al., Dis Model Mech 2020; 13.
39. Zhao et al., Mol Med 2019; 25:31.
40. Kaspar et al., Circ Cardiovasc Genet 2009; 2:544-551.
41. Kinder et al., Arthritis Rheumatol 2020; 72:1170-1183.
42. Heier et al., EMBO Mol Med 2013; 5:1569-1585.
43. von Haehling et al., J Cachexia Sarcopenia Muscle 2021; 12:2259-2261.

All references cited herein are incorporated by reference.

### Example 2 Additional Mouse Model of BMD Mutations

In addition to the BMD mouse model created by exon 45-47 deletion, such as the one described in detail in Example 1, this example provides a number of additional mouse models of BMD harboring alternate mutations. Some of these mutations include primarily internal deletions in the dystrophin gene, made either in wild type mice or as an additional mutation or a series of mutations made on top of the dystrophin exon 45-47 deletion BMD model mouse described herein.

Mice with these alternative mutations also model phenotypes of BMD, though they may present differing severities of the disease. It is unclear, from very heterogeneous clinical data and incomplete protein data, which mutations directly cause varying degrees of disease in patients. While not wishing to be bound by any particular theory, it is believed that different mutations differentially impact protein structure, protein stability, and/or protein interactions, and that mutations that cause a greater disruption of these aspects result in worse disease phenotypes.

Because certain mutations in this series of mutations result in milder disease, it may be possible to predict certain therapeutics by comparing mouse models featuring differing mutations and/or different disease severity. For example, exon skipping antisense oligos may be used to delete additional exons at the RNA level, or gene editing therapeutics may be used to delete additional exons at the DNA level, and either of these strategies may affect the structure of the encoded dystrophin protein isoform. If a therapeutic strategy converts a more severe BMD 45-47 model mouse genotype to a milder phenotype, the therapeutic strategy represents an efficacious therapy.

Additionally, different mouse models described herein may be used to determine if specific genotypes respond to various treatments or test compounds for BMD the same way or differently. For example, mice or patients with one mutation may respond better to a certain drug than those with another mutation. Alternatively, mice with one specific mutation may respond unfavorably or worse to a certain drug than those with another mutation. This could be a result of changes to the dystrophin protein itself, or changes to the interactions of dystrophin with other proteins.

While not intented to be limiting, the following additional mouse models with additional or alternative mutations in DNA sequences, which are analogous to those that cause BMD in humans, are made. Illustrative embodiments of these mutations include, but are not limited to:

Deletions starting at exon 45:
exon 45-48 deletion
exon 45-49 deletion
exon 45-51 deletion
exon 45-53 deletion
exon 45-55 deletion

Deletions of single exons:
exon 48 deletion
any single exon deletion between exon 23 and exon 42

In-frame deletions of other “hot spot” region exons
43-44
48-51
48-53
49-51
49-53
50-51
51-52
52-53
52-55

1. A mouse comprising an artificially (e.g., in-frame) deleted (e.g., not naturally existing) dystrophin/Dmd gene, such as completely or partially lacking exons 45-47 of a wild-type Dmd gene, wherein said artificially deleted dystrophin/Dmd gene leads to a BMD phenotype.

2. The mouse of claim 1, wherein the artificially deleted Dmd gene corresponds to the most common BMD mutation in human.

3. The mouse of claim 1 or 2, wherein the artificially deleted Dmd gene comprises a deletion of about 40,000 bp of genomic DNA sequence.

4. The mouse of any one of claims 1-3, wherein the artificially deleted Dmd gene comprises a deletions starting at exon 45, such as:
1) an exon 45-48 deletion;
2) an exon 45-49 deletion;
3) an exon 45-51 deletion;
4) an exon 45-53 deletion; or,
5) an exon 45-55 deletion.

5. The mouse of any one of claims 1-4, wherein the artificially deleted Dmd gene comprises a deletion of a single exon, such as:
1) an exon 48 deletion, or,
2) any single exon deletion between exon 23 and exon 42.

6. The mouse of any one of claims 1-5, wherein the artificially deleted Dmd gene comprises an in-frame deletion a hot-spot region exon selected from: exon 43-44, exon 48-51, exon 48-53, exon 49-51, exon 49-53, exon 50-51, exon 51-52, exon 52-53, and exon 52-55.

7. The mouse of any one of claims 1-6, wherein the artificially deleted Dmd gene is created via a DNase (e.g., a DNase capable of or designed for deleting said exons 45-47).

8. The mouse of any one of claims 1-7, wherein the DNase is a CRISPR/Cas effector enzyme (e.g., a Class 2, Type II enzyme such as Cas9, or a Class 2, Type V enzyme such as Cas12a/Cpf1, Cas12b, Cas12c, Cas12d, cas12e, Cas12f, Cas12g, Cas12h, Cas12i or Cas12k); a meganuclease, a ZFN (zinc finger nuclease), a TALEN (Transcription Activator-Like Effector Nuclease), an ARCUT (Artificial Restriction DNA Cutteror), or a Fok-dCas nuclease.

9. The mouse of any one of claims 1-8, which exhibits a phenotype intermediate to WT and mdx mice at the functional, molecular, and histological level.

10. The mouse of any one of claims 1-9, which shows impaired motor function, such as reduced forelimb and hindlimb grip strength (e.g., −15% or −39%), wire hang time, and/or in vivo isometric torque (e.g., −10%).

11. The mouse of any one of claims 1-10, wherein muscles from the mouse have increased myofiber size variability (minimal Feret's diameter) and centrally located nuclei that indicate of degeneration/regeneration.

12. The mouse of any one of claims 1-11, wherein muscles from the mouse have moderately increased levels of inflammatory/necrotic foci, collagen deposition (e.g., +1.4-fold or more) and/or trends of sarcolemmal damage as measured by the intracellular presence of IgM.

13. The mouse of any one of claims 1-12, wherein muscles from the mouse have reduced dystrophin protein levels (e.g., ˜20-50% of WT levels) in skeletal and cardiac muscles, and/or increased expression of miRNAs that target the 3′ UTR of Dmd transcripts; optionally, Dmd transcript levels are unchanged.

14. The mouse of any one of claims 1-13, wherein muscles from the mouse show increased expression of NF-kB-driven inflammatory genes and miRNAs, and genes indicative of active fibrosis.

15. The mouse of any one of claims 1-14, wherein serum from the mouse shows increased levels of creatine kinase (CK) compared to wt mouse, indicative of muscle damage.

16. The mouse of any one of claims 1-15, wherein the mouse shows signs of cardiomyopathy or decreased heart function compared to wt mouse, indicative of heart damage.

17. The mouse of any one of claims 1-16, which is homozygous/hemizygous.

18. The mouse of any one of claims 1-17, which is a male.

19. The mouse of any one of claims 1-17, which is a female.

20. The mouse of any one of claims 1-19, which is aged/aging (e.g., at least 3 months, 6 months, 9 months, 12 months, 15 months, 18 months, 21 months, 24 months, 27 months, or 30 months).

21. A muscle tissue of the mouse of any one of claims 1-20.

22. The muscle tissue of claim 21, which is a skeletal muscle tissue.

23. The muscle tissue of claim 21, which is a cardiac muscle tissue.

24. The muscle tissue of claim 21, which is a smooth muscle tissue.

25. A muscle cell of the mouse of any one of claims 1-20.

26. The muscle cell of claim 25, which is a primary cell.

27. The muscle cell of claim 25, which is a progeny of a cultured cell.

28. A method of characterizing BMD disease progression, the method comprising recording, analyzing, and/or characterizing at least one phenotype related to muscular dystrophy in the mouse of any one of claims 1-20, the muscle tissue of any one of claims 21-24, and/or the muscle cell of any one of claims 25-27.

29. The method of claim 28, further comprising applying a candidate therapy to the mouse, the muscle tissue, and/or the muscle cell.

30. The method of claim 29, wherein the candidate therapy is a small molecule compound, a biologic macromolecule (e.g., antisense polynucleotide), or a viral vector (e.g., a recombinant AAV expressing a gene effective to treat muscular dystrophy such as DMD and/or BMD).
